(19) United States
(12) Patent Application Publication
(10) Pub. No.: US 2016/0230230 A1
(43) Pub. Date: Aug. 11, 2016

(54) METHODS AND KITS FOR DIAGNOSING ULCERATIVE COLITIS IN A SUBJECT

(71) Applicants: INSERM (INSTITUT NATIONAL DE LA SANTÉ ET DE LA RECHERCHE MÉDICALE), Paris (FR); UNIVERSITÉ PARIS DIDEROT - PARIS 7, Paris (FR); UNIVERSITÉ DE VERSAILLES SAINT-QUENTIN-EN-YVELINES, Versailles (FR); ASSISTANCE PUBLIQUE-HÔPITAUX DE PARIS (APHP), Paris (FR)

(72) Inventors: Eric Ogier-Denis, Paris (FR); Yannick Ladeiro, Paris (FR); Xavier Treton, Paris (FR); Yoram Bouhnik, Paris (FR); Yong-Ping Ding, Paris (FR)

(21) Appl. No.: 15/024,997

(22) PCT Filed: Sep. 26, 2014
(86) PCT No.: PCT/EP2014/070595

§ 371 Date: Mar. 25, 2016

(30) Foreign Application Priority Data
Sep. 26, 2013 (EP) 13306314.9

(51) Int. Cl. C12Q 1/68

(52) U.S. Cl. CPC C12Q 1/6883; C12Q 2600/178; C12Q 2600/158

(57) ABSTRACT

The present invention relates to methods and kits for diagnosing ulcerative colitis in a subject. In particular, the present invention relates to a method for diagnosing ulcerative colitis in a subject comprising the steps consisting of determining in a sample obtained from the subject the expression level of at least one gene selected from the group consisting of ADH4, ADH6, ADHFE1, AKR1A1, AKR7A2, ALDH1A3, ALDH1L1, ALDH7A1, AOX1, BCHE, CBR3, CES1, CYP1B1, CYP2E1, CYP2W1, CYP4F11, CYP51A1, ESD, KCNAB2, COMT, GSTA4, GSTP1, INMT, MGST2, SULT2A1, TPMT, UGT1A4, UGT1A9, UGT2B7, ABCA1, ABCA2, ABCB1, ABCC1, ABCC10, ABCC5, ABCC6, ABCG2, ATP7A, SLC1A3, SLC7A5, SLC10A2, SLC15A1, SLC15A2, SLC19A2, SLC19A3, SLC22A3, SLC28A3, SLC29A2, SLC38A1, SLC38A5, SLC47A1, SLCO2B1, SLCO4C1, ARNT, FOXO1, HIF3A, NCOA2, NCOR2, NR1H3, NR3C1, PPARD, PPARGC1A, RARB, RXRB, and THRB.

### FIELD OF THE INVENTION

The present invention relates to methods and kits for diagnosing ulcerative colitis in a subject.

### BACKGROUND OF THE INVENTION

Ulcerative colitis (UC) is an idiopathic, chronic inflammatory bowel disease (IBD) of the colonic mucosa which starts in the rectum and generally extends proximally in a continuous manner. In clinical practice, 20 to 30% of patients with IBD colitis cannot be classified as Crohn's disease (CD) or UC based upon usual endoscopic, radiologic, and histopathologic criteria, though this distinction may be crucial to guide therapeutic choices, especially when colonic resection is discussed. Similarly, the sensitivity of serological markers (autoantibodies to neutrophils [ANCA, pANCA] and antimicrobial antibodies [ASCA, anti-OmpC, anti-I2, and anti-CBir1]) remains insufficient to discriminate between CD and UC. On the other hand, the aetiology of IBDs and the cause of flare still remain largely unknown and specific biomarkers of UC are also needed to assess an early diagnosis. To date, strictly specific biomarkers remain difficult to elect.

The precise cause of UC is unknown; however, several environmental factors have been implicated including smoking, xenobiotics, diet, and microbial agents. The most indisputable example of the influence of the environment on IBD is cigarette smoking (CS). Smoking has a striking opposite effect on UC and CD [1]. While cigarette use is an important risk factor for CD, patients with UC are frequently non-smokers and cessation of smoking increases the risk of developing UC, supporting the notion that distinct mechanisms underlie the pathogenesis of each form of IBD. However, the protective mechanisms of CS on UC are still obscure.

Human xenobiotic-metabolizing enzyme machinery is a major protective factor from environmental exposition [2]. Although the liver is the major organ for detoxification, colonic epithelial cells have an equal capacity to detoxify luminal environmental factors [3, 4]. The failure of detoxification capacity of harmful luminal agents may be seen as an important factor of the pathogenesis of UC. For instance, few data from animal model of colitis [5, 6] and previous studies in patients with UC [7] as well as others in IBD [3, 4, 8, 9, 10] suggest that detoxification enzyme depletion may be involved in the initiation and progression of colitis. However, information provided by these studies on the concept of a multilevel alteration of the detoxification system leading to a weak responsiveness of the epithelial barrier to environmental exposure is limited and most often concerns a small number of genes.

### SUMMARY OF THE INVENTION

The present invention relates to methods and kits for diagnosing ulcerative colitis in a subject. In particular, the present invention is defined by the claims.

### DETAILED DESCRIPTION OF THE INVENTION

Evidence suggests that xenobiotic metabolism may play a role in inflammatory bowel disease (IBD). The global expression profile of 244 genes encoding detoxification machinery was studied in inactive colonic samples from patients with ulcerative colitis (UC) compared with healthy controls and patients with Crohn's disease (CD). The inventors identified a set of 65 detoxification genes significantly deregulated in patients with UC. Thirty percent and 63% of them were inversely expressed in control and UC active smoking groups, respectively.

Accordingly a first object of the present invention relates to a method for diagnosing ulcerative colitis in a subject comprising the steps consisting of i) determining in a sample obtained from the subject the expression level of at least one gene selected from the group consisting of ADH4, ADH6, ADHFE1, AKR1A1, AKR7A2, ALDH1A3, ALDH1L1, ALDH7A1, AOX1, BCHE, CBR3, CES1, CYP1B1, CYP2E1, CYP2W1, CYP4F11, CYP51A1, ESD, KCNAB2, COMT, GSTA4, GSTP1, INMT, MGST2, SULT2A1, TPMT, UGT1A4, UGT1A9, UGT2B7, ABCA1, ABCA2, ABCB1, ABCC1, ABCC10, ABCC5, ABCC6, ABCG2, ATP7A, SLC1A3, SLC7A5, SLC10A2, SLC15A1, SLC15A2, SLC19A2, SLC19A3, SLC22A3, SLC28A3, SLC29A2, SLC38A1, SLC38A5, SLC47A1, SLCO2B1, SLCO4C1, ARNT, FOXO1, HIF3A, NCOA2, NCOR2, NR1H3, NR3C1, PPARD, PPARGC1A, RARB, RXRB, and THRB, ii) comparing the expression level determined at step i) with a reference value and iii) concluding that the subject suffers from ulcerative colitis when
the expression determined at step i) is higher than the reference value for each gene selected from the group consisting of ADH6, AKR1A1, AKR7A2, ALDH1L1, ALDH7A1, CBR3, CES1, CYP51A1, ESD, KCNAB2, COMT, GSTA4, GSTP1, MGST2, UGT2B17, ABCC1, SLC28A3, SLC29A1, SLC38A1, and SLC38A5, or
the expression determined at step at step i) is lower than the reference value for each gene selected from the group consisting of ADH4, ADHFE1, ALDH1A3, AOX1, BCHE, CYP1B1, CYP2E1, CYP2W1, CYP4F11, INMT, SULT2A1, TPMT, UGT1A4, UGT1A9, ABCA1, ABCA2, ABCB1, ABCC10, ABCC5, ABCC6, ABCG2, ATP7A, SLC1A3, SLC7A5, SLC10A2, SLC15A1, SLC15A2, SLC19A2, SLC19A3, SLC22A3, SLC47A1, SLCO2B1, SLCO4C1, ARNT, FOXO1, HIF3A, NCOA2, NCOR2, NR1H3, NR3C1, PPARD, PPARGC1A, RARB, RXRB, and THRB

The term “sample” means any sample derived from the colon of the patient, which comprises mucosal cells. Said sample is obtained for the purpose of the in vitro evaluation. In a particular embodiment the sample results from an endoscopical biopsy performed in the colon of the patient. Said endoscopical biopsy may be taken from various areas of the colon. In another particular embodiment, the sample may be isolated from non-inflamed mucosa of the patient's colon. Consequently, the invasiveness of the method according to the invention is relatively limited without the need of anesthetizing the patient or of purging the patient's intestines.

As used herein the term “ADH4” has its general meaning in the art and refers to the gene encoding for alcohol dehydrogenase 4 (class II), pi polypeptide.

As used herein the term “ADH6” has its general meaning in the art and refers to the gene encoding for alcohol dehydrogenase 6 (class V).

As used herein the term “ADHFE1” has its general meaning in the art and refers to the gene encoding for hydroxyacid-oxoacid transhydrogenase (EC 1 1.99.24).

As used herein the term “AKR1A1” has its general meaning in the art and refers to the gene encoding for aldo-keto reductase family 1, member A1.

As used herein the term “AKR7A2” has its general meaning in the art and refers to the gene encoding for aldo-keto reductase family 7, member A2 (aflatoxin aldehyde reductase).

As used herein the term “ALDH1A3” has its general meaning in the art and refers to the gene encoding for aldehyde dehydrogenase 1 family, member A3.

As used herein the term “ALDH1L1” has its general meaning in the art and refers to the gene encoding for aldehyde dehydrogenase 1 family, member L1.

As used herein the term “ALDH7A1” has its general meaning in the art and refers to the gene encoding for aldehyde dehydrogenase 7 family, member A1.

As used herein the term “AOX1” has its general meaning in the art and refers to the gene encoding for aldehyde oxidase 1.

As used herein the term “BCHE” has its general meaning in the art and refers to the gene encoding for butyrylcholinesterase.

As used herein the term “CBR3” has its general meaning in the art and refers to the gene encoding for carbonyl reductase 3.

As used herein the term “CES1” has its general meaning in the art and refers to the gene encoding for carboxylesterase 1.

As used herein the term “CYP1B1” has its general meaning in the art and refers to the gene encoding for cytochrome P450, family 1, subfamily B, polypeptide 1.

As used herein the term “CYP2E” has its general meaning in the art and refers to the gene encoding for cytochrome P450, family 2, subfamily E, polypeptide 1.

As used herein the term “CYP2W1” has its general meaning in the art and refers to the gene encoding for cytochrome P450, family 2, subfamily W, polypeptide 1.

As used herein the term “CYP4F11” has its general meaning in the art and refers to the gene encoding for cytochrome P450, family 4, subfamily F, polypeptide 11.

As used herein the term “CYP51A1” has its general meaning in the art and refers to the gene encoding for cytochrome P450, family 51, subfamily A, polypeptide 1.

As used herein the term “ESD” has its general meaning in the art and refers to the gene encoding for esterase D.

As used herein the term “KCNAB2” has its general meaning in the art and refers to the gene encoding for potassium voltage-gated channel, shaker-related subfamily, beta member 2.

As used herein the term “COMT” has its general meaning in the art and refers to the gene encoding for catechol-O-methyltransferase.

As used herein the term “GSTA4” has its general meaning in the art and refers to the gene encoding for glutathione S-transferase alpha 4.

As used herein the term “GSTP1” has its general meaning in the art and refers to the gene encoding for glutathione S-transferase pi 1.

As used herein the term “INMT” has its general meaning in the art and refers to the gene encoding for indolethylamine N-methyltransferase.

As used herein the term “MGST2” has its general meaning in the art and refers to the gene encoding for microsomal glutathione S-transferase 2.

As used herein the term “SULT2A1” has its general meaning in the art and refers to the gene encoding for sulfotransferase family, cytosolic, 2A, dehydroepiandrosterone (DHEA)-preferring, member 1.

As used herein the term “TPMT” has its general meaning in the art and refers to the gene encoding for thiopurine S-methyltransferase.

As used herein the term “UGT1A4” has its general meaning in the art and refers to the gene encoding for UDP glucuronosyltransferase 1 family, polypeptide A4.

As used herein the term “UGT1A9” has its general meaning in the art and refers to the gene encoding for UDP glucuronosyltransferase 1 family, polypeptide A9.

As used herein the term “UGT2B7” has its general meaning in the art and refers to the gene encoding for UDP glucuronosyltransferase 2 family, polypeptide B7.

As used herein the term “ABCA1” has its general meaning in the art and refers to the gene encoding for ATP-binding cassette, sub-family A (ABC1), member 1.

As used herein the term “ABCA2” has its general meaning in the art and refers to the gene encoding for ATP-binding cassette, sub-family A (ABC1), member 2.

As used herein the term “ABCB1” has its general meaning in the art and refers to the gene encoding for ATP-binding cassette, sub-family B (MDR/TAP), member 1.

As used herein the term “ABCC1” has its general meaning in the art and refers to the gene encoding for ATP-binding cassette, sub-family C (CFTR/MRP), member 1.

As used herein the term “ABCC10” has its general meaning in the art and refers to the gene encoding for ATP-binding cassette, sub-family C (CFTR/MRP), member 10.

As used herein the term “ABCC5” has its general meaning in the art and refers to the gene encoding for ATP-binding cassette, sub-family C (CFTR/MRP), member 5.

As used herein the term “ABCC6” has its general meaning in the art and refers to the gene encoding for ATP-binding cassette, sub-family C (CFTR/MRP), member 6.

As used herein the term “ABCG2” has its general meaning in the art and refers to the gene encoding for ATP-binding cassette, sub-family G (WHITE), member 2.

As used herein the term “ATP7A” has its general meaning in the art and refers to the gene encoding for ATPase, Cu++ transporting, alpha polypeptide.

As used herein the term “SLC1A3” has its general meaning in the art and refers to the gene encoding for solute carrier family 1 (glial high affinity glutamate transporter), member 3.

As used herein the term “SLC7A5” has its general meaning in the art and refers to the gene encoding for solute carrier family 7 (amino acid transporter light chain, L system), member 5.

As used herein the term “SLC10A2” has its general meaning in the art and refers to the gene encoding for solute carrier family 10 (sodium/bile acid cotransporter), member 2.

As used herein the term “SLC15A1” has its general meaning in the art and refers to the gene encoding for solute carrier family 15 (oligopeptide transporter), member 1.

As used herein the term “SLC15A2” has its general meaning in the art and refers to the gene encoding for solute carrier family 19 (thiamine transporter), member 2.

As used herein the term “SLC19A2” has its general meaning in the art and refers to the gene encoding for solute carrier family 19 (thiamine transporter), member 2.

As used herein the term “SLC19A3” has its general meaning in the art and refers to the gene encoding for solute carrier family 19 (thiamine transporter), member 3.

As used herein the term “SLC22A3” has its general meaning in the art and refers to the gene encoding for solute carrier family 22 (organic cation transporter), member 3.

As used herein the term “SLC28A3” has its general meaning in the art and refers to the gene encoding for solute carrier family 28 (concentrative nucleoside transporter), member 3.

As used herein the term “SLC29A2” has its general meaning in the art and refers to the gene encoding for solute carrier family 29 (equilibrative nucleoside transporter), member 2.

As used herein the term “SLC38A1” has its general meaning in the art and refers to the gene encoding for solute carrier family 38, member 1.

As used herein the term “SLC38A5” has its general meaning in the art and refers to the gene encoding for solute carrier family 38, member 5.

As used herein the term “SLC47A1” has its general meaning in the art and refers to the gene encoding for solute carrier family 47 (multidrug and toxin extrusion), member 1.

As used herein the term “SLCO2B1” has its general meaning in the art and refers to the gene encoding for solute carrier organic anion transporter family, member 2B1.

As used herein the term “SLCO4C1” has its general meaning in the art and refers to the gene encoding for solute carrier organic anion transporter family, member 4C1.

As used herein the term “ARNT” has its general meaning in the art and refers to the gene encoding for aryl hydrocarbon receptor nuclear translocator.

As used herein the term “FOXO1” has its general meaning in the art and refers to the gene encoding for forkhead box 01.

As used herein the term “HIF3A” has its general meaning in the art and refers to the gene encoding for hypoxia inducible factor 3, alpha subunit.

As used herein the term “NCOA2” has its general meaning in the art and refers to the gene encoding for nuclear receptor coactivator 2.

As used herein the term “NCOR2” has its general meaning in the art and refers to the gene encoding for nuclear receptor corepressor 2.

As used herein the term “NR1H3” has its general meaning in the art and refers to the gene encoding for nuclear receptor subfamily 1, group H, member 3.

As used herein the term “NR3C1” has its general meaning in the art and refers to the gene encoding for nuclear receptor subfamily 3, group C, member 1 (glucocorticoid receptor).

As used herein the term “PPARD” has its general meaning in the art and refers to the gene encoding for peroxisome proliferator-activated receptor delta.

As used herein the term “PPARGC1A” has its general meaning in the art and refers to the gene encoding for peroxisome proliferator-activated receptor gamma, coactivator 1 alpha.

As used herein the term “RARB” has its general meaning in the art and refers to the gene encoding for retinoic acid receptor, beta.

As used herein the term “RXRB” has its general meaning in the art and refers to the gene encoding for retinoid X receptor, beta.

As used herein the term “THRB” has its general meaning in the art and refers to the gene encoding for thyroid hormone receptor, beta.

In some embodiments, the expression level of 1; 2; 3; 4; 5; 6; 7; 8; 9; 10; 11; 12; 13; 14; 15; 16; 17; 18; 19; 20; 21; 22; 23; 24; 25; 26; 27; 28; 29; 30; 31; 32; 33; 34; 35; 36; 37; 38; 39; 40; 41; 42; 43; 44; 45; 46; 47; 48; 49; 50; 51; 52; 53; 54; 55; 56; 57; 58; 59; 60; 61; 62; 63; 64 or 65 genes is(are) determined.

One skilled in the art may easily select the appropriate method for determining the expression level of the gene.

Typically, the expression level of a gene may be determined by determining the quantity of mRNA. Methods for determining the quantity of mRNA are well known in the art. For example the nucleic acid contained in the samples (e.g., cell or tissue prepared from the patient) is first extracted according to standard methods, for example using lytic enzymes or chemical solutions or extracted by nucleic-acid-binding resins following the manufacturer's instructions. The extracted mRNA is then detected by hybridization (e. g., Northern blot analysis, in situ hybridization) and/or amplification (e.g., RT-PCR).

Other methods of Amplification include ligase chain reaction (LCR), transcription-mediated amplification (TMA), strand displacement amplification (SDA) and nucleic acid sequence based amplification (NASBA).

Nucleic acids having at least 10 nucleotides and exhibiting sequence complementarity or homology to the mRNA of interest herein find utility as hybridization probes or amplification primers. It is understood that such nucleic acids need not be identical, but are typically at least about 80% identical to the homologous region of comparable size, more preferably 85% identical and even more preferably 90-95% identical. In certain embodiments, it will be advantageous to use nucleic acids in combination with appropriate means, such as a detectable label, for detecting hybridization.

Typically, the nucleic acid probes include one or more labels, for example to permit detection of a target nucleic acid molecule using the disclosed probes. In various applications, such as in situ hybridization procedures, a nucleic acid probe includes a label (e.g., a detectable label). A “detectable label” is a molecule or material that can be used to produce a detectable signal that indicates the presence or concentration of the probe (particularly the bound or hybridized probe) in a sample. Thus, a labeled nucleic acid molecule provides an indicator of the presence or concentration of a target nucleic acid sequence (e.g., genomic target nucleic acid sequence) (to which the labeled uniquely specific nucleic acid molecule is bound or hybridized) in a sample. A label associated with one or more nucleic acid molecules (such as a probe generated by the disclosed methods) can be detected either directly or indirectly. A label can be detected by any known or yet to be discovered mechanism including absorption, emission and/or scattering of a photon (including radio frequency, microwave frequency, infrared frequency, visible frequency and ultra-violet frequency photons). Detectable labels include colored, fluorescent, phosphorescent and luminescent molecules and materials, catalysts (such as enzymes) that convert one substance into another substance to provide a detectable difference (such as by converting a colorless substance into a colored substance or vice versa, or by producing a precipitate or increasing sample turbidity), haptens that can be detected by antibody binding interactions, and paramagnetic and magnetic molecules or materials.

Particular examples of detectable labels include fluorescent molecules (or fluorochromes). Numerous fluorochromes are known to those of skill in the art, and can be selected, for example from Life Technologies (formerly Invitrogen), e.g., see, The Handbook—A Guide to Fluorescent Probes and Labeling Technologies). Examples of particular fluorophores that can be attached (for example, chemically conjugated) to a nucleic acid molecule (such as a uniquely specific binding region) are provided in U.S. Pat. No. 5,866,366 to Nazarenko et al., such as 4-acetamido-4′-isothiocyanatostilbene-2,2′ disulfonic acid, acridine and derivatives such as acridine and acridine isothiocyanate, 5-(2′-aminoethyl) aminonaphthalene-1-sulfonic acid (EDANS), 4-amino-N-[³ vinylsulfonyl)phenyl]naphthalimide-3,5 disulfonate (Lucifer Yellow VS), N-(4-anilino-1-naphthyl)maleimide, antl1ranilamide, Brilliant Yellow, coumarin and derivatives such as coumarin, 7-amino-4-methylcoumarin (AMC, Coumarin 120), 7-amino-4-trifluoromethylcouluarin (Coumarin 151); cyanosine; 4′,6-diarninidino-2-phenylindole (DAPI); 5′,5″dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfor1ic acid; 5-[dimethylamino]naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); eosin and derivatives such as eosin and eosin isothiocyanate; erythrosin and derivatives such as erythrosin B and erythrosin isothiocyanate; ethidium; fluorescein and derivatives such as 5-carboxyfluorescein (FAM), 5-(4,6dicl1lorotriazin-2-yDarninofluorescein (DTAF), 2′7′dimethoxy-4′5′-dichloro-6-carboxyfluorescein (JOE), fluorescein, fluorescein isothiocyanate (FITC), and QFITC Q(RITC); 2′,7′-difluorofluorescein (OREGON GREEN®); fluorescamine; IR144; IR1446; Malachite Green isothiocyanate; 4-methylumbelliferone; ortho cresolphthalein; nitrotyrosine; pararosaniline; Phenol Red; B-phycoerythrin; o-phthaldialdehyde; pyrene and derivatives such as pyrene, pyrene butyrate and succinimidyl 1-pyrene butyrate; Reactive Red 4 (Cibacron Brilliant Red 3B-A); rhodamine and derivatives such as 6-carboxy-X-rhodamine (ROX), 6-carboxyrhodamine (R6G), lissamine rhodamine B sulfonyl chloride, rhodamine (Rhod), rhodamine B, rhodamine 123, rhodamine X isothiocyanate, rhodamine green, sulforhodamine B, sulforhodamine 101 and sulfonyl chloride derivative of sulforhodamine 101 (Texas Red); N,N,N′,N′-tetramethyl-6-carboxyrhodamine (TAMRA); tetramethyl rhodamine; tetramethyl rhodamine isothiocyanate (TRITC); riboflavin; rosolic acid and terbium chelate derivatives. Other suitable fluorophores include thiol-reactive europium chelates which emit at approximately 617 mn (Heyduk and Heyduk, Analyt. Biochem. 248:216-27, 1997; J. Biol. Chem. 274:3315-22, 1999), as well as GFP, Lissamine™, diethylaminocoumarin, fluorescein chlorotriazinyl, naphthofluorescein, 4,7-dichlororhodamine and xanthene (as described in U.S. Pat. No. 5,800,996 to Lee et al.) and derivatives thereof. Other fluorophores known to those skilled in the art can also be used, for example those available from Life Technologies (Invitrogen; Molecular Probes (Eugene, Oreg.)) and including the ALEXA FLUOR® series of dyes (for example, as described in U.S. Pat. Nos. 5,696,157, 6,130,101 and 6,716,979), the BODIPY series of dyes (dipyrrometheneboron difluoride dyes, for example as described in U.S. Pat. Nos. 4,774,339, 5,187,288, 5,248,782, 5,274,113, 5,338,854, 5,451,663 and 5,433,896), Cascade Blue (an amine reactive derivative of the sulfonated pyrene described in U.S. Pat. No. 5,132,432) and Marina Blue (U.S. Pat. No. 5,830,912).

In addition to the fluorochromes described above, a fluorescent label can be a fluorescent nanoparticle, such as a semiconductor nanocrystal, e.g., a QUANTUM DOT™ (obtained, for example, from Life Technologies (QuantumDot Corp, Invitrogen Nanocrystal Technologies, Eugene, Oreg.); see also, U.S. Pat. Nos. 6,815,064; 6,682,596; and 6,649, 138). Semiconductor nanocrystals are microscopic particles having size-dependent optical and/or electrical properties. When semiconductor nanocrystals are illuminated with a primary energy source, a secondary emission of energy occurs of a frequency that corresponds to the handgap of the semiconductor material used in the semiconductor nanocrystal. This emission can he detected as colored light of a specific wavelength or fluorescence. Semiconductor nanocrystals with different spectral characteristics are described in e.g., U.S. Pat. No. 6,602,671. Semiconductor nanocrystals that can he coupled to a variety of biological molecules (including dNTPs and/or nucleic acids) or substrates by techniques described in, for example, Bruchez et al., Science 281:20132016, 1998; Chan et al., Science 281:2016-2018, 1998; and U.S. Pat. No. 6,274,323. Formation of semiconductor nanocrystals of various compositions are disclosed in, e.g., U.S. Pat. Nos. 6,927,069; 6,914,256; 6,855,202; 6,709,929; 6,689,338; 6,500,622; 6,306,736; 6,225,198; 6,207,392; 6,114,038; 6,048,616; 5,990,479; 5,690,807; 5,571,018; 5,505,928; 5,262,357 and in U.S. Patent Publication No. 2003/0165951 as well as PCT Publication No. 99/26299 (published May 27, 1999). Separate populations of semiconductor nanocrystals can he produced that are identifiable based on their different spectral characteristics. For example, semiconductor nanocrystals can he produced that emit light of different colors hased on their composition, size or size and composition. For example, quantum dots that emit light at different wavelengths based on size (565 mn, 655 mn, 705 mn, or 800 mn emission wavelengths), which are suitable as fluorescent labels in the probes disclosed herein are available from Life Technologies (Carlshad, Calif.).

Additional labels include, for example, radioisotopes (such as 3H), metal chelates such as DOTA and DPTA chelates of radioactive or paramagnetic metal ions like Gd3+, and liposomes.

Detectable labels that can he used with nucleic acid molecules also include enzymes, for example horseradish peroxidase, alkaline phosphatase, acid phosphatase, glucose oxidase, beta-galactosidase, beta-glucuronidase, or beta-lactamase.

Alternatively, an enzyme can he used in a metallographic detection scheme. For example, silver in situ hyhridization (SISH) procedures involve metallographic detection schemes for identification and localization of a hybridized genomic target nucleic acid sequence. Metallographic detection methods include using an enzyme, such as alkaline phosphatase, in combination with a water-soluble metal ion and a redox-inactive substrate of the enzyme. The substrate is converted to a redox-active agent by the enzyme, and the redoxactive agent reduces the metal ion, causing it to form a detectable precipitate. (See, for example, U.S. Patent Application Publication No. 2005/0100976, PCT Publication No. 2005/003777 and U.S. Patent Application Publication No. 2004/0265922). Metallographic detection methods also include using an oxido-reductase enzyme (such as horseradish peroxidase) along with a water soluble metal ion, an oxidizing agent and a reducing agent, again to form a detectable precipitate. (See, for example, U.S. Pat. No. 6,670,113).

Probes made using the disclosed methods can be used for nucleic acid detection, such as ISH procedures (for example, fluorescence in situ hybridization (FISH), chromogenic in situ hybridization (CISH) and silver in situ hybridization (SISH)) or comparative genomic hybridization (CGH).

In situ hybridization (ISH) involves contacting a sample containing target nucleic acid sequence (e.g., genomic target nucleic acid sequence) in the context of a metaphase or interphase chromosome preparation (such as a cell or tissue sample mounted on a slide) with a labeled probe specifically hybridizable or specific for the target nucleic acid sequence (e.g., genomic target nucleic acid sequence). The slides are optionally pretreated, e.g., to remove paraffin or other materials that can interfere with uniform hybridization. The sample and the probe are both treated, for example by heating to denature the double stranded nucleic acids. The probe (formulated in a suitable hybridization buffer) and the sample are combined, under conditions and for sufficient time to permit hybridization to occur (typically to reach equilibrium). The chromosome preparation is washed to remove excess probe, and detection of specific labeling of the chromosome target is performed using standard techniques.

For example, a biotinylated probe can be detected using fluorescein-labeled avidin or avidin-alkaline phosphatase. For fluorochrome detection, the fluorochrome can be detected directly, or the samples can be incubated, for example, with fluorescein isothiocyanate (FITC)-conjugated avidin. Amplification of the FITC signal can be effected, if necessary, by incubation with biotin-conjugated goat antiavidin antibodies, washing and a second incubation with FITC-conjugated avidin. For detection by enzyme activity, samples can be incubated, for example, with streptavidin, washed, incubated with biotin-conjugated alkaline phosphatase, washed again and pre-equilibrated (e.g., in alkaline phosphatase (AP) buffer). For a general description of in situ hybridization procedures, see, e.g., U.S. Pat. No. 4,888,278.

Numerous procedures for FISH, CISH, and SISH are known in the art. For example, procedures for performing FISH are described in U.S. Pat. Nos. 5,447,841; 5,472,842; and 5,427,932; and for example, in Pir1kel et al., Proc. Natl. Acad. Sci. 83:2934-2938, 1986; Pinkel et al., Proc. Natl. Acad. Sci. 85:9138-9142, 1988; and Lichter et al., Proc. Natl. Acad. Sci. 85:9664-9668, 1988. CISH is described in, e.g., Tanner et al., Am. 0.1. Pathol. 157:1467-1472, 2000 and U.S. Pat. No. 6,942,970. Additional detection methods are provided in U.S. Pat. No. 6,280,929.

Numerous reagents and detection schemes can be employed in conjunction with FISH, CISH, and SISH procedures to improve sensitivity, resolution, or other desirable properties. As discussed above probes labeled with fluorophores (including fluorescent dyes and QUANTUM DOTS®) can be directly optically detected when performing FISH. Alternatively, the probe can be labeled with a nonfluorescent molecule, such as a hapten (such as the following non-limiting examples: biotin, digoxigenin, DNP, and various oxazoles, pyrrazoles, thiazoles, nitroaryls, benzofurazans, triterpenes, ureas, thioureas, rotenones, coumarin, courmarin-based compounds, Podophyllotoxin, Podophyllotoxin-based compounds, and combinations thereof), ligand or other indirectly detectable moiety. Probes labeled with such non-fluorescent molecules (and the target nucleic acid sequences to which they bind) can then be detected by contacting the sample (e.g., the cell or tissue sample to which the probe is bound) with a labeled detection reagent, such as an antibody (or receptor, or other specific binding partner) specific for the chosen hapten or ligand. The detection reagent can be labeled with a fluorophore (e.g., QUANTUM DOT®) or with another indirectly detectable moiety, or can be contacted with one or more additional specific binding agents (e.g., secondary or specific antibodies), which can be labeled with a fluorophore.

In other examples, the probe, or specific binding agent (such as an antibody, e.g., a primary antibody, receptor or other binding agent) is labeled with an enzyme that is capable of converting a fluorogenic or chromogenic composition into a detectable fluorescent, colored or otherwise detectable signal (e.g., as in deposition of detectable metal particles in SISH). As indicated above, the enzyme can be attached directly or indirectly via a linker to the relevant probe or detection reagent. Examples of suitable reagents (e.g., binding reagents) and chemistries (e.g., linker and attachment chemistries) are described in U.S. Patent Application Publication Nos. 2006/0246524; 2006/0246523, and 2007/01 17153.

It will he appreciated by those of skill in the art that by appropriately selecting labelled probe-specific binding agent pairs, multiplex detection schemes can he produced to facilitate detection of multiple target nucleic acid sequences (e.g., genomic target nucleic acid sequences) in a single assay (e.g., on a single cell or tissue sample or on more than one cell or tissue sample). For example, a first probe that corresponds to a first target sequence can he labelled with a first hapten, such as biotin, while a second probe that corresponds to a second target sequence can be labelled with a second hapten, such as DNP. Following exposure of the sample to the probes, the bound probes can he detected by contacting the sample with a first specific binding agent (in this case avidin labelled with a first fluorophore, for example, a first spectrally distinct QUANTUM DOT®, e.g., that emits at 585 mn) and a second specific binding agent (in this case an anti-DNP antibody, or antibody fragment, labelled with a second fluorophore (for example, a second spectrally distinct QUANTUM DOT®, e.g., that emits at 705 mn). Additional probes/binding agent pairs can he added to the multiplex detection scheme using other spectrally distinct fluorophores. Numerous variations of direct, and indirect (one step, two step or more) can he envisioned, all of which are suitable in the context of the disclosed probes and assays.

Probes typically comprise single-stranded nucleic acids of between 10 to 1000 nucleotides in length, for instance of between 10 and 800, more preferably of between 15 and 700, typically of between 20 and 500. Primers typically are shorter single-stranded nucleic acids, of between 10 to 25 nucleotides in length, designed to perfectly or almost perfectly match a nucleic acid of interest, to be amplified. The probes and primers are “specific” to the nucleic acids they hybridize to, i.e. they preferably hybridize under high stringency hybridization conditions (corresponding to the highest melting temperature Tm, e.g., 50% formamide, 5× or 6×SCC. SCC is a 0.15 M NaCl, 0.015 M Na-citrate).

The nucleic acid primers or probes used in the above amplification and detection method may be assembled as a kit. Such a kit includes consensus primers and molecular probes. A preferred kit also includes the components necessary to determine if amplification has occurred. The kit may also include, for example, PCR buffers and enzymes; positive control sequences, reaction control primers; and instructions for amplifying and detecting the specific sequences.

In a particular embodiment, the methods of the invention comprise the steps of providing total RNAs extracted from cumulus cells and subjecting the RNAs to amplification and hybridization to specific probes, more particularly by means of a quantitative or semi-quantitative RT-PCR.

In another preferred embodiment, the expression level is determined by DNA chip analysis. Such DNA chip or nucleic acid microarray consists of different nucleic acid probes that are chemically attached to a substrate, which can be a microchip, a glass slide or a microsphere-sized bead. A microchip may be constituted of polymers, plastics, resins, polysaccharides, silica or silica-based materials, carbon, metals, inorganic glasses, or nitrocellulose. Probes comprise nucleic acids such as cDNAs or oligonucleotides that may be about 10 to about 60 base pairs. To determine the expression level, a sample from a test subject, optionally first subjected to a reverse transcription, is labelled and contacted with the microarray in hybridization conditions, leading to the formation of complexes between target nucleic acids that are complementary to probe sequences attached to the microarray surface. The labelled hybridized complexes are then detected and can be quantified or semi-quantified.

Labelling may be achieved by various methods, e.g. by using radioactive or fluorescent labelling. Many variants of the microarray hybridization technology are available to the man skilled in the art (see e.g. the review by Hoheisel, Nature Reviews, Genetics, 2006, 7:200-210).

In some embodiments, the nCounter® Analysis system is used to detect intrinsic gene expression. The basis of the nCounter® Analysis system is the unique code assigned to each nucleic acid target to be assayed (International Patent Application Publication No. WO 08/124847, U.S. Pat. No. 8,415,102 and Geiss et al. Nature Biotechnology. 2008. 26(3): 317-325; the contents of which are each incorporated herein by reference in their entireties). The code is composed of an ordered series of colored fluorescent spots which create a unique barcode for each target to be assayed. A pair of probes is designed for each DNA or RNA target, a biotinylated capture probe and a reporter probe carrying the fluorescent barcode. This system is also referred to, herein, as the nanoreporter code system. Specific reporter and capture probes are synthesized for each target. The reporter probe can comprise at a least a first label attachment region to which are attached one or more label monomers that emit light constituting a first signal; at least a second label attachment region, which is non-over-lapping with the first label attachment region, to which are attached one or more label monomers that emit light constituting a second signal; and a first target-specific sequence. Preferably, each sequence specific reporter probe comprises a target specific sequence capable of hybridizing to no more than one gene and optionally comprises at least three, or at least four label attachment regions, said attachment regions comprising one or more label monomers that emit light, constituting at least a third signal, or at least a fourth signal, respectively. The capture probe can comprise a second target-specific sequence; and a first affinity tag. In some embodiments, the capture probe can also comprise one or more label attachment regions. Preferably, the first target-specific sequence of the reporter probe and the second target-specific sequence of the capture probe hybridize to different regions of the same gene to be detected. Reporter and capture probes are all pooled into a single hybridization mixture, the “probe library”. The relative abundance of each target is measured in a single multiplexed hybridization reaction. The method comprises contacting the tumor sample with a probe library, such that the presence of the target in the sample creates a probe pair-target complex. The complex is then purified. More specifically, the sample is combined with the probe library, and hybridization occurs in solution. After hybridization, the tripartite hybridized complexes (probe pairs and target) are purified in a two-step procedure using magnetic beads linked to oligonucleotides complementary to universal sequences present on the capture and reporter probes. This dual purification process allows the hybridization reaction to be driven to completion with a large excess of target-specific probes, as they are ultimately removed, and, thus, do not interfere with binding and imaging of the sample. All post hybridization steps are handled robotically on a custom liquid-handling robot (Prep Station, NanoString Technologies). Purified reactions are typically deposited by the Prep Station into individual flow cells of a sample cartridge, bound to a streptavidin-coated surface via the capture probe, electrophoresed to elongate the reporter probes, and immobilized. After processing, the sample cartridge is transferred to a fully automated imaging and data collection device (Digital Analyzer, NanoString Technologies). The expression level of a target is measured by imaging each sample and counting the number of times the code for that target is detected. For each sample, typically 600 fields-of-view (FOV) are imaged (1376×1024 pixels) representing approximately 10 mm2 of the binding surface. Typical imaging density is 100-1200 counted reporters per field of view depending on the degree of multiplexing, the amount of sample input, and overall target abundance. Data is output in simple spreadsheet format listing the number of counts per target, per sample. This system can be used along with nanoreporters. Additional disclosure regarding nanoreporters can be found in International Publication No. WO 07/076129 and WO07/076132, and US Patent Publication No. 2010/0015607 and 2010/0261026, the contents of which are incorporated herein in their entireties. Further, the term nucleic acid probes and nanoreporters can include the rationally designed (e.g. synthetic sequences) described in International Publication No. WO 2010/019826 and US Patent Publication No. 2010/0047924, incorporated herein by reference in its entirety.

Expression level of a gene may be expressed as absolute expression level or normalized expression level. Typically, expression levels are normalized by correcting the absolute expression level of a gene by comparing its expression to the expression of a gene that is not a relevant, e.g., a housekeeping gene that is constitutively expressed. Suitable genes for normalization include housekeeping genes such as the actin gene ACTB, ribosomal 18S gene, GUSB, PGK1 and TFRC. This normalization allows the comparison of the expression level in one sample, e.g., a patient sample, to another sample, or between samples from different sources.

Other methods for determining the expression level of a gene include the determination of the quantity of proteins encoded by said genes.

Such methods comprise contacting the sample with a binding partner capable of selectively interacting with a marker protein present in the sample. The binding partner is generally an antibody that may be polyclonal or monoclonal, preferably monoclonal. The binding partner may also be an aptamer.

The presence of the protein can be detected using standard electrophoretic and immunodiagnostic techniques, including immunoassays such as competition, direct reaction, or sandwich type assays. Such assays include, but are not limited to, Western blots; agglutination tests; enzyme-labeled and mediated immunoassays, such as ELISAs; biotin/avidin type assays; radioimmunoassays; immunoelectrophoresis; immunoprecipitation, etc. The reactions generally include revealing labels such as fluorescent, chemiluminescent, radioactive, enzymatic labels or dye molecules, or other methods for detecting the formation of a complex between the antigen and the antibody or antibodies reacted therewith.

The aforementioned assays generally involve separation of unbound protein in a liquid phase from a solid phase support to which antigen-antibody complexes are bound. Solid supports which can be used in the practice of the invention include substrates such as nitrocellulose (e. g., in membrane or microtiter well form); polyvinylchloride (e. g., sheets or microtiter wells); polystyrene latex (e.g., beads or microtiter plates); polyvinylidine fluoride; diazotized paper; nylon membranes; activated beads, magnetically responsive beads, and the like.

More particularly, an ELISA method can be used, wherein the wells of a microtiter plate are coated with an antibody against the protein to be tested. A biological sample containing or suspected of containing the marker protein is then added to the coated wells. After a period of incubation sufficient to allow the formation of antibody-antigen complexes, the plate (s) can be washed to remove unbound moieties and a detectably labeled secondary binding molecule added. The secondary binding molecule is allowed to react with any captured sample marker protein, the plate washed and the presence of the secondary binding molecule detected using methods well known in the art.

Alternatively an immunohistochemistry (IHC) method may be preferred. IHC specifically provides a method of detecting targets in a sample or tissue specimen in situ. The overall cellular integrity of the sample is maintained in IHC, thus allowing detection of both the presence and location of the targets of interest. Typically a sample is fixed with formalin, embedded in paraffin and cut into sections for staining and subsequent inspection by light microscopy. Current methods of IHC use either direct labeling or secondary antibody-based or hapten-based labeling. Examples of known IHC systems include, for example, EnVision™ (DakoCytomation), Powervision® (Immunovision, Springdale, Ariz.), the NBA™ kit (Zymed Laboratories Inc., South San Francisco, Calif.), HistoFine® (Nichirei Corp, Tokyo, Japan).

In particular embodiment, a tissue section (e.g. a sample comprising cumulus cells) may be mounted on a slide or other support after incubation with antibodies directed against the proteins encoded by the genes of interest. Then, microscopic inspections in the sample mounted on a suitable solid support may be performed. For the production of photomicrographs, sections comprising samples may be mounted on a glass slide or other planar support, to highlight by selective staining the presence of the proteins of interest.

Therefore IHC samples may include, for instance: (a) preparations comprising cumulus cells (b) fixed and embedded said cells and (c) detecting the proteins of interest in said cells samples. In some embodiments, an IHC staining procedure may comprise steps such as: cutting and trimming tissue, fixation, dehydration, paraffin infiltration, cutting in thin sections, mounting onto glass slides, baking, deparaffination, rehydration, antigen retrieval, blocking steps, applying primary antibodies, washing, applying secondary antibodies (optionally coupled to a suitable detectable label), washing, counter staining, and microscopic examination.

In some embodiments, the method of the present invention further comprises a step consisting of i) determining the expression level of at least one miRNA selected from the group consisting of miR15a, miR26a, miR29a, miR29b, miR30c, miR126*, miR127-3p, miR-142-3p, miR-142-5p, miR-146a, miR-146b-5p, miR150, miR-181d, miR-182, miR185, miR196a, miR199a-3p, miR199a-5p, miR199b-5p, miR-203, miR223, miR-299-5p, miR320a, miR324-3p, and miR-328, ii) comparing the expression level determined at step i) with a reference value and iii) concluding that the subject suffers from an ulcerative disease when
the expression determined at step i) is higher than the reference value for each miRNA selected from the group consisting of miR15a, miR26a, miR29a, miR29b, miR30c, miR126*, miR127-3p, miR185, miR196a, miR324-3p, and miR-146b-5p
the expression determined at step at step i) is lower than the reference value for each miRNA selected from the group consisting of miR150, miR-181d, miR-182, miR199a-3p, miR199a-5p, miR199b-5p, miR-203, miR223, miR-299-5p, miR320a, miR-146a, miR-142-3p, miR-142-5p, and miR-328.

The term “miRNAs” refers to mature microRNA (non-coding small RNAs) molecules that are generally 21 to 22 nucleotides in length, even though lengths of 19 and up to 23 nucleotides have been reported. miRNAs are each processed from longer precursor RNA molecules (“precursor miRNA”: pri-miRNA and pre-miRNA). Pri-miRNAs are transcribed either from non-protein-encoding genes or embedded into protein-coding genes (within introns or non-coding exons). The “precursor miRNAs” fold into hairpin structures containing imperfectly base-paired stems and are processed in two steps, catalyzed in animals by two Ribonuclease III-type endonucleases called Drosha and Dicer. The processed miRNA is typically a portion of the stem. The processed miRNAs (also referred to as “mature miRNA”) are assembled into large ribonucleoprotein complexes (miRISCs) that post-transcriptional repression (down-regulation) of a specific target gene(s). All the miRNAs pertaining to the invention are known per se and sequences of them are publicly available from the data base http://www.mirbase.org/cgi-bin/mirna_summary.pl?org=hsa. The miRNAs of the invention are listed in Table A:

| miRNA | Accession_Number |
|---|---|
| hsa-mir-15a | MIMAT0000068 |
| hsa-mir-26a | MIMAT0000082 |
| hsa-mir-29a | MIMAT0000086 |
| hsa-mir-29b | MIMAT0000100 |
| hsa-mir-30c | MIMAT0000244 |
| hsa-mir-126* | MIMAT0000444 |
| hsa-mir-127-3p | MIMAT0000446 |
| hsa-mir-146a | MIMAT0000449 |
| hsa-mir-150 | MIMAT0000451 |
| hsa-mir-142-5p | MIMAT0000433 |
| hsa-mir-142-3p | MIMAT0000434 |
| hsa-mir-146b-5p | MIMAT0002809 |
| hsa-mir-181d | MIMAT0002821 |
| hsa-mir-182 | MIMAT0000259 |
| hsa-mir-185 | MIMAT0000455 |
| hsa-mir-196a | MIMAT0000226 |
| hsa-mir-199a-3p | MIMAT0000232 |
| hsa-mir-199a-5p | MIMAT0000231 |
| hsa-mir-199b-5p | MIMAT0000263 |
| hsa-mir-203 | MIMAT0000264 |
| hsa-mir-223 | MIMAT0000280 |
| hsa-mir-299-5p | MIMAT0002890 |
| hsa-mir-320 | MIMAT0000510 |
| hsa-mir-324-3p | MIMAT0000762 |
| hsa-mir-328 | MIMAT0000752 |

In some embodiments, the expression level of 1, 2; 3; 4; 5; 6; 7; 8; 9; 10; 11; 12; 13; 14; 15; 16; 17; 18; 19; 20; 21; 22; 23; 24; 25 miRNA is determined.

As used herein the term “reference value” refers to the value determined for the gene or miRNA in population of healthy subjects. “Healthy subjects” denote subjects who do not suffer from an ulcerative disease, and more preferably from an inflammatory bowel disease. Typically the reference value is chosen in order to obtain the optimal sensitivity and specificity, i.e. the benefice/risk balance (clinical consequences of false positive and false negative). For example, the optimal sensitivity and specificity (and so the reference value) can be determined using a Receiver Operating Characteristic (ROC) curve based on experimental data obtained form a test cohort of subjects.

Once the subject is diagnosed with ulcerative colitis, he could be treated with at least one compound selected from the group consisting of:
analgesics: morphine, fentanyl, hydromorphone, oxycodone, codeine, acetaminophen, hydrocodone, buprenorphine, tramadol, venlafaxine, flupirtine, meperidine, pentazocine, dextromoramide, dipipanone;
antibiotics—aminoglycosides (e.g., amikacin, gentamicin, kanamycin, neomycin, netilmicin, tobramycin, and paromycin), carbapenems (e.g., ertapenem, doripenem, imipenem, cilastatin, and meropenem), cephalosporins (e.g., cefadroxil, cefazolin, cefalotin, cephalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, and cefobiprole), glycopeptides (e.g., teicoplanin, vancomycin, and telavancin), lincosamides (e.g., clindamycin and incomysin), lipopeptides) e.g., daptomycin), macro lides (azithromycin, clarithromycin, dirithromycin, erythromycin, roxithromycin, troleandomycin, telithromycin, and spectinomycin), monobactams (e.g., aztreonam), nitrofurans (e.g., furazolidone and nitrofurantoin), penicilllins (e.g., amoxicillin, ampicillin, azlocillin, carbenicillin, cloxacillin, dicloxacillin, flucloxacillin, mezlocillin, methicillin, nafcillin, oxacillin, penicillin G, penicillin V, piperacillin, temocillin, and ticarcillin), penicillin combinations (e.g., amoxicillin/clavulanate, ampicillin/sulbactam, piperacillin/tazobactam, and ticarcillin/clavulanate), polypeptides (e.g., bacitracin, colistin, and polymyxin B), quinolones (e.g., ciprofloxacin, enoxacin, gatifloxacin, levofloxacin, lomefloxacin, moxifloxacin, nalidixic acid, norfloxacin, ofloxacin, trovafloxacin, grepafloxacin, sparfloxacin, and temafloxacin), sulfonamides (e.g., mafenide, sulfonamidochrysoidine, sulfacetamide, sulfadiazine, silver sulfadiazine, sulfamethizole, sulfamethoxazole, sulfanamide, sulfasalazine, sulfisoxazole, trimethoprim, and trimethoprim-sulfamethoxaxzole), tetracyclines (e.g., demeclocycline, doxycycline, minocycline, oxytetracycline, and tetracycline), antimycobacterial compounds (e.g., clofazimine, dapsone, capreomycin, cycloserine, ethambutol, ethionamide, isoniazid, pyrazinamide, rifampicin (rifampin), rifabutin, rifapentine, and streptomycin), and others, such as arsphenamine, chloramphenicol, fosfomycin, fusidic acid, linezolid, metronidazole, mupirocin, platensimycin, quinuprisin/dalfopristin, rifaximin, thiamphenicol, tigecycline, and tinidazole;
antibodies—anti-TNF-a antibody, e.g., infliximab (Remicade®);
anticoagulants—warfarin (Coumadin®), acenocoumarol, phenprocoumon, atromentin, phenindione, heparin, fondaparinux, idraparinux, rivaroxaban, apixaban, hirudin, lepirudin, bivalirudin, argatrobam, dabigatran, ximelagatran, batroxobin, hementin;
anti-inflammatory agents—steroids, e.g., budesonide, nonsteroidal anti-inflammatory agents, e.g., aminosalicylates (e.g., sulfasalazine, mesalamine, olsalazine, and balsalazide), cyclooxygenase inhibitors (COX-2 inhibitors, such as rofecoxib, celecoxib), diclofenac, etodolac, famotidine, fenoprofen, flurbiprofen, ketoprofen, ketorolac, ibuprofen, indomethacin, meclofenamate, mefenamic acid, meloxicam, nambumetone, naproxen, oxaprozin, piroxicam, salsalate, sulindac, tolmetin;
aminosalicylates sulfasalazine, such as Mesalazine (also known as 5-aminosalicylic acid, mesalamine, or 5-ASA. Brand name formulations include Apriso, Asacol, Pentasa, Mezavant, Lialda, Fivasa, Rovasa and Salofalk), Sulfasalazine (also known as Azulfidine), Balsalazide (also known as Colazal or Colazide (UK)), Olsalazine (also known as Dipentum),
immunosuppressants—mercaptopurine, corticosteroids such as dexamethasone, hydrocortisone, prednisone, methylprednisolone and prednisolone, alkylating agents such as cyclophosphamide, calcineurin inhibitors such as cyclosporine, sirolimus and tacrolimus, inhibitors of inosine monophosphate dehydrogenase (IMPDH) such as mycophenolate, mycophenolate mofetil and azathioprine, and agents designed to suppress cellular immunity while leaving the recipient's humoral immunologic response intact, including various antibodies (for example, antilymphocyte globulin (ALG), antithymocyte globulin (ATG), monoclonal anti-T-cell antibodies (OKT3)) and irradiation. Azathioprine is currently available from Salix Pharmaceuticals, Inc. under the brand name Azasan®; mercaptopurine is currently available from Gate Pharmaceuticals, Inc. under the brand name Purinethol®; prednisone and prednisolone are currently available from Roxane Laboratories, Inc.; Methyl prednisolone is currently available from Pfizer; sirolimus (rapamycin) is currently available from Wyeth-Ayerst under the brand name Rapamune®; tacrolimus is currently available from Fujisawa under the brand name Prograf®; cyclosporine is current available from Novartis under the brand dame Sandimmune® and Abbott under the brand name Gengraf®; IMPDH inhibitors such as mycophenolate mofetil and mycophenolic acid are currently available from Roche under the brand name Cellcept® and Novartis under the brand name Myfortic®; azathioprine is currently available from Glaxo Smith Kline under the brand name Imuran®; and antibodies are currently available from Ortho Biotech under the brand name Orthoclone®, Novartis under the brand name Simulect® (basiliximab) and Roche under the brand name Zenapax® (daclizumab).
Guanylate cyclase-C receptor agonists or intestinal secretagogues—for example linaclotide, sold under the name Linzess®.
Sulforaphane, guanabenz and salubrinal

These various agents can be used in accordance with their standard or common dosages, as specified in the prescribing information accompanying commercially available forms of the drugs.

A further object relates to a chip comprising a solid support which carries at least one nucleic acid specific for at least one gene selected from the group consisting of ADH4, ADH6, ADHFE1, AKR1A1, AKR7A2, ALDH1A3, ALDH1L1, ALDH7A1, AOX1, BCHE, CBR3, CES1, CYP1B1, CYP2E1, CYP2W1, CYP4F11, CYP51A1, ESD, KCNAB2, COMT, GSTA4, GSTP1, INMT, MGST2, SULT2A1, TPMT, UGT1A4, UGT1A9, UGT2B7, ABCA1, ABCA2, ABCB1, ABCC1, ABCC10, ABCC5, ABCC6, ABCG2, ATP7A, SLC1A3, SLC7A5, SLC10A2, SLC15A1, SLC15A2, SLC19A2, SLC19A3, SLC22A3, SLC28A3, SLC29A2, SLC38A1, SLC38A5, SLC47A1, SLCO2B1, SLCO4C1, ARNT, FOXO1, HIF3A, NCOA2, NCOR2, NR1H3, NR3C1, PPARD, PPARGC1A, RARB, RXRB, and THRB.

In some embodiments, the solid support of the chip carries a set of nucleic acids specific for 2; 3; 4; 5; 6; 7; 8; 9; 10; 11; 12; 13; 14; 15; 16; 17; 18; 19; 20; 21; 22; 23; 24; 25; 26; 27; 28; 29; 30; 31; 32; 33; 34; 35; 36; 37; 38; 39; 40; 41; 42; 43; 44; 45; 46; 47; 48; 49; 50; 51; 52; 53; 54; 55; 56; 57; 58; 59; 60; 61; 62; 63; 64 or 65 genes.

In some embodiments, the solid support of the chip carries at least one specific nucleic acid specific for at least one miRNA selected from the group consisting of miR15a, miR26a, miR29a, miR29b, miR30c, miR126*, miR127-3p, miR-142-3p, miR-142-5p, miR-146a, miR-146b-5p, miR150, miR-181d, miR-182, miR185, miR196a, miR199a-3p, miR199a-5p, miR199b-5p, miR-203, miR223, miR-299-5p, miR320a, miR324-3p, and miR-328.

In some embodiments, solid support of the chip carries a set of nucleic acids specific for 1, 2; 3; 4; 5; 6; 7; 8; 9; 10; 11; 12; 13; 14; 15; 16; 17; 18; 19; 20; 21; 22; 23; 24; 25 miRNA.

Accordingly such a chip (or nucleic acid microarray) consists of different nucleic acid probes that are chemically attached to the support, which can be a microchip, a glass slide or a microsphere-sized bead. A microchip may be constituted of polymers, plastics, resins, polysaccharides, silica or silica-based materials, carbon, metals, inorganic glasses, or nitrocellulose. Probes comprise nucleic acids such as cDNAs or oligonucleotides that may be about 10 to about 60 base pairs. To determine the expression level, a sample from a test subject, optionally first subjected to a reverse transcription, is labelled and contacted with the microarray in hybridization conditions, leading to the formation of complexes between target nucleic acids that are complementary to probe sequences attached to the microarray surface. The labelled hybridized complexes are then detected and can be quantified or semi-quantified. Labelling may be achieved by various methods, e.g. by using radioactive or fluorescent labelling. Many variants of the microarray hybridization technology are available to the man skilled in the art (see e.g. the review by Hoheisel, Nature Reviews, Genetics, 2006, 7:200-210).

A further object of the present invention relates to a kit comprising means for determining the expression level of at least one gene selected from the group consisting of ADH4, ADH6, ADHFE1, AKR1A1, AKR7A2, ALDH1A3, ALDH1L1, ALDH7A1, AOX1, BCHE, CBR3, CES1, CYP1B1, CYP2E1, CYP2W1, CYP4F11, CYP51A1, ESD, KCNAB2, COMT, GSTA4, GSTP1, INMT, MGST2, SULT2A1, TPMT, UGT1A4, UGT1A9, UGT2B7, ABCA1, ABCA2, ABCB1, ABCC1, ABCC10, ABCC5, ABCC6, ABCG2, ATP7A, SLC1A3, SLC7A5, SLC10A2, SLC15A1, SLC15A2, SLC19A2, SLC19A3, SLC22A3, SLC28A3, SLC29A2, SLC38A1, SLC38A5, SLC47A1, SLCO2B1, SLCO4C1, ARNT, FOXO1, HIF3A, NCOA2, NCOR2, NR1H3, NR3C1, PPARD, PPARGC1A, RARB, RXRB, and THRB.

In some embodiments, the kit comprises means for determining the expression of 1; 2; 3; 4; 5; 6; 7; 8; 9; 10; 11; 12; 13; 14; 15; 16; 17; 18; 19; 20; 21; 22; 23; 24; 25; 26; 27; 28; 29;

30; 31; 32; 33; 34; 35; 36; 37; 38; 39; 40; 41; 42; 43; 44; 45; 46; 47; 48; 49; 50; 51; 52; 53; 54; 55; 56; 57; 58; 59; 60; 61; 62; 63; 64 or 65 genes.

In some embodiments, the kit further comprises means for determining the level of least one miRNA selected from the group consisting of miR15a, miR26a, miR29a, miR29b, miR30c, miR126*, miR127-3p, miR-142-3p, miR-142-5p, miR-146a, miR-146b-5p, miR150, miR-181d, miR-182, miR185, miR196a, miR199a-3p, miR199a-5p, miR199b-5p, miR-203, miR223, miR-299-5p, miR320a, miR324-3p, and miR-328.

In some embodiments, the kit comprises means for determining the expression level of 1, 2; 3; 4; 5; 6; 7; 8; 9; 10; 11; 12; 13; 14; 15; 16; 17; 18; 19; 20; 21; 22; 23; 24; 25 miRNA.

For example, the kit may comprise a primer set for amplifying a target sequence in the gene or miRNA. In certain embodiments, the primer set contains primer pairs (forward and reverse primers) for amplifying the gene or miRNA. In certain embodiments, the kit further comprises a primer set for amplifying at least one normalization gene, such as one or more normalization genes described herein. Additionally, the kit may comprise at least one probe for detecting each target sequence, including in connection with the detection platforms described herein (e.g., TaqMan™).

Kits, may comprise containers, each with one or more of the various reagents (sometimes in concentrated form), for example, pre-fabricated microarrays, buffers, the appropriate nucleotide triphosphates (e.g., dATP, dCTP, dGTP and dTTP; or rATP, rCTP, rGTP and UTP), reverse transcriptase, DNA polymerase, RNA polymerase, and one or more primer complexes (e.g., appropriate length poly(T) or random primers linked to a promoter reactive with the RNA polymerase). A set of instructions will also typically be included.

In some embodiments, the kit may comprise a plurality of reagents, each of which is capable of binding specifically with a target nucleic acid or protein. Suitable reagents for binding with a target protein include antibodies, antibody derivatives, antibody fragments, and the like. Suitable reagents for binding with a target nucleic acid (e.g. a genomic DNA, an mRNA, a spliced mRNA, a cDNA, or the like) include complementary nucleic acids. For example, nucleic acid reagents may include oligonucleotides (labeled or non-labeled) fixed to a substrate, labeled oligonucleotides not bound with a substrate, pairs of PCR primers, molecular beacon probes, and the like.

In some embodiments, the kit may comprise additional components useful for detecting gene expression levels. By way of example, the kit may comprise fluids (e.g. SSC buffer) suitable for annealing complementary nucleic acids or for binding an antibody with a protein with which it specifically binds, one or more sample compartments, a material which provides instruction for detecting expression levels, and the like.

In some embodiments, one or more of the primers is “linear”. A “linear” primer refers to an oligonucleotide that is a single stranded molecule, and typically does not comprise a short region of, for example, at least 3, 4 or 5 contiguous nucleotides, which are complementary to another region within the same oligonucleotide such that the primer forms an internal duplex. In some embodiments, the primers for use in reverse transcription comprise a region of at least 4, such as at least 5, such as at least 6, such as at least 7 or more contiguous nucleotides at the 3′-end that has a base sequence that is complementary to region of at least 4, such as at least 5, such as at least 6, such as at least 7 or more contiguous nucleotides at the 5′-end of a target RNA. In some embodiments, the kit further comprises one or more pairs of linear primers (a “forward primer” and a “reverse primer”) for amplification of a cDNA reverse transcribed from a target RNA. Accordingly, in some embodiments, the forward primer comprises a region of at least 4, such as at least 5, such as at least 6, such as at least 7, such as at least 8, such as at least 9, such as at least 10 contiguous nucleotides having a base sequence that is complementary to the base sequence of a region of at least 4, such as at least 5, such as at least 6, such as at least 7, such as at least 8, such as at least 9, such as at least 10 contiguous nucleotides at the 5′-end of a target RNA. Furthermore, in some embodiments, the reverse primer comprises a region of at least 4, such as at least 5, such as at least 6, such as at least 7, such as at least 8, such as at least 9, such as at least 10 contiguous nucleotides having a base sequence that is complementary to the base sequence of a region of at least 4, such as at least 5, such as at least 6, such as at least 7, such as at least 8, such as at least 9, such as at least 10 contiguous nucleotides at the 3′-end of a target RNA.

In some embodiments, the kit comprises a set of antibodies to each of the protein products of the genes of the invention, conjugated to a detectable substance, and instructions for use. The kit may comprise an antibody, an antibody derivative, or an antibody fragment, which binds specifically with a marker protein, or a fragment of the protein. Such the kit may also comprise a plurality of antibodies, antibody derivatives, or antibody fragments wherein the plurality of such antibody agents binds specifically with a marker protein, or a fragment of the protein.

In some embodiments, the kit may comprise antibodies such as a labeled or labelable antibody and a compound or agent for detecting protein in a biological sample; means for determining the amount of protein in the sample; means for comparing the amount of protein in the sample with a standard; and instructions for use. Such kits can be supplied to detect a single protein or epitope or can be configured to detect one of a multitude of epitopes, such as in an antibody detection array. Arrays are described in detail herein for nucleic acid arrays and similar methods have been developed for antibody arrays.

A person skilled in the art will appreciate that a number of detection agents can be used to determine the expression of the biomarkers. For example, to detect RNA products of the biomarkers, probes, primers, complementary nucleotide sequences or nucleotide sequences that hybridize to the RNA products can be used. To detect protein products of the biomarkers, ligands or antibodies that specifically bind to the protein products can be used.

A person skilled in the art will appreciate that the detection agents can be labeled. The label is preferably capable of producing, either directly or indirectly, a detectable signal. For example, the label may be radio-opaque or a radioisotope, such as ³H, ¹⁴C, ³²P, ³⁵S, ¹²³I, ¹²⁵I, ¹³¹I; a fluorescent (fluorophore) or chemiluminescent (chromophore) compound, such as fluorescein isothiocyanate, rhodamine or luciferin; an enzyme, such as alkaline phosphatase, beta-galactosidase or horseradish peroxidase; an imaging agent; or a metal ion.

The kit can also include a set of reference values and/or instructions for use thereof. In addition, the kit can include ancillary agents such as vessels for storing or transporting the detection agents and/or buffers or stabilizers.

The invention will be further illustrated by the following figures and examples. However, these examples and figures should not be interpreted in any way as limiting the scope of the present invention.

### EXAMPLE

### Material and Methods

Patients and Biopsies

Human ascending colon biopsies were obtained from the IBD Gastroenterology Unit, Hôpital Beaujon. The protocol was in agreement with local Ethics Committee (CPP-Ile de France IV No. 2009/17) and written informed consent was obtained from all patients before enrollment. The clinical characteristics of patients with UC were shown in supplementary table 1. Nineteen non-smoking and 3 smoking patients with UC, 8 non-smoking and 8 smoking controls and 20 patients with CD (10 with Crohn's ileocolitis and 10 with Crohn's colitis) were selected and included in this study. All diagnoses of patients were based on classical clinical features as well as radiologic, endoscopic, and histological findings. All biopsies were picked in non-affected right colon to avoid variability of detoxification enzyme expression along the colon. Unaffected areas were defined as mucosa regions without any macroscopic/endoscopic and histologic signs of inflammation. To preserve the transcriptional profiles of tissue specimens, biopsy specimens were immediately kept in −80° C. until RNA extraction.

Isolation of Total mRNA and Reverse Transcription

Total mRNA was extracted from the human ascending colon biopsies using RNAble® Kit (Eurobio Courtaboeuf, France). RNA was quantified by ND-1000 NanoDrop spectrophotometer (NanoDrop technologies Inc., France) and integrity of total mRNA was verified by Agilent 2100 Bionanalyser. Total mRNA (1 μg) was converted to cDNA using Moloney Murine Leukemia Virus Reverse Transcriptase (M-MLV RT) kit (Invitrogen, Carlsbad, Calif., USA) according to manufacturer protocol. The reverse-transcription was achieved using Thermal Cyclers (Mastercycler®, Eppendorf, Germany).

Quantitative PCR

Real-time quantitative PCR was performed with SYBR Green (Mastermix plus for SYBR® assay No ROX, Eurogentec, USA) using the Lightcycler 480 system (Roche, France). Cycling conditions were as follows: 10 min at 95° C., followed by 50 cycles of 15 s at 95° C., 1 min at 65° C., followed by 5 s at 95° C. and 1 min at 55° C. After the 50 cycles a melting curve (10 min) at 40° C. was performed. The melting curve was analyzed with the Lightcycler® 480 gene scanning software. Obtained cycle threshold (Ct) of the target genes were normalized for those of housekeeping genes as TATA box binding protein (TBP). The method 2^{-ΔΔCt} was used to calculate the fold induction of target genes. The sequences of nucleotides were obtained from the literatures^{[1-5]} or home designed using the primer designing tool (NCBI Primer-Blast).

Statistical Analysis

Statistical analysis was performed using Prism Version 5.0 (GraphPad software, Inc., San Diego, Calif.). Mann-Whitney test was used to determine statistical significance between groups. As the sample of smoking patients with UC is limited, there is no suitable test for this group. Values were considered statistically different when p<0.05. Results are presented as mean±SEM. Clustering was performed using dChip software.

Results:

Ulcerative colitis (UC) is an idiopathic, chronic inflammatory bowel disease (IBD) of the colonic mucosa which starts in the rectum and generally extends proximally in a continuous manner. The precise cause of UC is unknown; however, several environmental factors have been implicated including smoking, xenobiotics, diet, and microbial agents. The most indisputable example of the influence of the environment on IBD is cigarette smoking (CS). Smoking has a striking opposite effect on UC and CD [1]. While cigarette use is an important risk factor for CD, patients with UC are frequently non-smokers and cessation of smoking increases the risk of developing UC, supporting the notion that distinct mechanisms underlie the pathogenesis of each form of IBD. However, the protective mechanisms of CS on UC are still obscure.

Human xenobiotic-metabolizing enzyme machinery is a major protective factor from environmental exposition [2]. Although the liver is the major organ for detoxification, colonic epithelial cells have an equal capacity to detoxify luminal environmental factors [3, 4]. The failure of detoxification capacity of harmful luminal agents may be seen as an important factor of the pathogenesis of UC. For instance, few data from animal model of colitis [5, 6] and our previous studies in patients with UC [7] as well as others in IBD [3, 4, 8, 9, 10] suggest that detoxification enzyme depletion may be involved in the initiation and progression of colitis. However, information provided by these studies on the concept of a multilevel alteration of the detoxification system leading to a weak responsiveness of the epithelial barrier to environmental exposure is limited and most often concerns a small number of genes.

Here, we performed comprehensive and integrated investigations of xenobiotic detoxification capacity of non-affected colonic mucosa from patients with UC to enable a better understanding of the susceptibility of this tissue to environmental aggression and its implication in the pathogenesis. We have mainly investigated whether beneficial protective effect of CS in UC could be explained in part by its regulatory effect on the expression of detoxification enzymes.

Gene expression of 244 detoxification enzymes, including phase I and phase 2 xenobiotic-metabolizing enzymes (XMEs), transporters, and nuclear receptors and transcription factors, known to be expressed in human gastrointestinal tract [3] was quantified by qRT-PCR in individual non-inflamed colonic biopsies picked in the right colon from 19 patients with UC and 8 healthy controls (Supplementary Tables 1 and 2). Results showed that 65 genes assigned to 3 different subgroups: XMEs, ABC or SLC transporters, and nuclear receptors were significantly deregulated in patients with UC compared to healthy subjects (fold change >|1, 5|, P value <0.05). Of these genes, 70% (45/65) were down-regulated (Supplementary Table 2). We noted a specific down-regulation of transcription factors and nuclear receptors when compare to others subgroups of detoxification genes (Fisher's exact test P=0.003) (Supplementary Table 2). Using the gene function prediction tool Genemania (http://genemania.org/) we identified different deregulated clusters of coordinately regulated genes belonging to the aryl hydrocarbon receptor AhR (including cofactors ARNT, NCOA2, NCOR2, NR3C1 and a set of downstream targets genes ABCB1, ABCG2, ALDH1A3, ALDH7A1, AOX1, COMT, CYP1B1, CYP2E1, CYP2W1, INMT, UGT1A4, UGT1A9, SULT2A1, SLC7A5) and pregnane-X-receptor PXR/NR1I2 pathways (ABCB1/MDR1, ABCC1, SULT2A1), and fatty acid metabolism (i.e. PPARs, NR1H3 or LXR, RXR), known to be for some of them deregulated in IBD and animal models [5, 10, 11, 12, 13].

Hierarchical clustering analysis identified two distinct clusters based on the similarity of the 65 detoxification gene expression profiles, clearly separating patients with UC (P=0.02) and controls (P=0.009). The detoxification gene expression profile was next analysed in non-inflamed colonic mucosa from 20 patients with CD (10 patients with ileocolitis and 10 patients with colitis). Patients with CD and healthy controls were not statistically separable from gene profiles with only 15/65 genes exhibiting a similar expression profile in patients with UC (Supplementary Table 3). These data pointed out for a specific deregulation of detoxification gene expression in the non-affected colonic epithelial mucosa from patients with UC.

Regarding the protective effect of CS on UC, we investigated its regulatory effect on the 65 detoxification gene expression in colonic mucosa from 9 smoking and 9 non-smoking controls. Interestingly, we found that 28 genes out of 65 were differentially up or down-regulated by CS in controls. Among these genes, 15 XMEs (including CYP1B1, CYP2W1, TPMT, SULT2A1), 7 transporters (including ABCC1, SLC15A2, SLC47A1) and 6 nuclear receptors and transcription factors (HIF3A, NCOA2, PPARD, PPARGC1A, RARB, NR1H3). Interestingly, the majority of them (71%, 20/28) was inversely expressed in patients with UC (Supplementary Table 4). These data clearly support the idea that smoking may affect per se the colonic detoxification gene expression and provide new avenues about the impact of CS on UC. However, this observation only concerns non-IBD patients who exhibit any deregulation in the detoxification machinery. It seemed interesting to directly evaluate the effect of CS on patients with UC. We have had the opportunity to obtain rare colonic biopsies of three patients with UC being in clinical, endoscopic, and histological remission following smoking resumption (Supplementary Table 1). Although the weak number of patients impaired statistical power of our analysis, expression of the 65 deregulated genes in smoking patients with quiescent UC was quantified. Similarity and differences in gene expression levels between the different groups (non-smoking and smoking controls and patients with UC) were illustrated by principal component analysis. This analysis reveals the high degree of similarity in gene expression levels between the smoking patients with UC and smoking control patients. This result suggests that CS robustly counter-regulates altered detoxification gene expression in the colon of patients with UC gathering the smoking control and UC groups. Interestingly, 40 of the 65 deregulated genes seen in UC were inversely expressed by CS reaching to similar level of expression observed in control groups (Supplementary Table 4). Many of these genes belonging to the transporter family were induced by CS exposure. ABC and SCL transporters have an important role in tissue defence through the excretion of toxic compounds and their metabolites protecting the colonic epithelium. Likewise, nuclear receptors and transcription factors which are overarching regulators of the xenobiotic response system including detoxification enzymes and transporters were strongly up-regulated by CS. One hypothesis could be that increased toxicity induced by CS in the colon would be able to activate the expression of detoxification genes. This activation would achieve a protective threshold level of expression allowing the colonic mucosa to better support and detoxify chemicals endogenous or exogenous agents.

In conclusion, we identified a specific deregulation of the xenobiotic detoxification system in non-inflamed colonic mucosa from patients with UC establishing a clear cut gene signature for UC that could help a better diagnosis of UC. Interestingly, we found that CS modulated detoxification gene expression and could help normalizing this deregulation of gene expression essential to the colon detoxification of xenobiotic and luminal agents. The pathophysiology relevance in UC for those genes is actually explored in our laboratory and open promising new targets for developing CS mimicking-therapies in UC.

SUPPLEMENTARY TABLE 1
Patient's clinical characteristics
Patients with Crohn Disease

| | Age | | | | | | | | | | | |
|---|---|---|---|---|---|---|---|---|---|---|---|---|
| | at | | Cumulative | | | Disease | | | | | | |
| | biopsy | | disease | Cigarette | Disease | duration | Cortico | 5asa | | Imurel | Imurel | Other |
| Patient | (years) | Sex | extension | smoker | activity | (years) | dependance | treatment | Cholangitis | treatment | failure | treatments |
| MICI023 | 19 | F | pancolitis | yes | Active | 2 | yes | no | no | no | yes | anti-TNF |
| MICI024 | 31 | F | ileo- | former | Active | 11 | yes | no | no | no | yes | anti-TNF |
| | | | colitis | | | | | | | | | |
| MICI027 | 62 | F | iléo- | yes | Quiescent | 10 | no | yes | no | no | no | |
| | | | colitis | | | | | | | | | |
| MICI029 | 18 | F | colitis | former | Quiescent | 1 | yes | no | no | no | no | anti-TNF |
| MICI032 | 31 | M | ileo- | no | Active | 1 | no | no | no | yes | yes | |
| | | | colitis | | | | | | | | | |
| MICI103 | 53 | F | pancolitis | no | Quiescent | 30 | yes | no | no | yes | no | |
| MICI142 | 28 | M | ileo- | former | Quiescent | 1 | no | no | yes | no | no | anti-TNF |
| | | | colitis | | | | | | | | | |
| MICI144 | 45 | M | colitis | former | Active | 11 | yes | no | no | intolerent | intolerent | anti-TNF |
| MICI145 | 59 | M | colitis | former | Quiescent | 8 | no | no | no | no | no | |
| MICI146 | 31 | M | ileo- | no | Active | 10 | no | no | no | no | yes | |
| | | | colitis | | | | | | | | | |
| MICI147 | 44 | F | colitis | yes | Active | 19 | yes | no | no | no | no | anti-TNF |
| MICI148 | 25 | M | ileo- | no | Active | 8 | no | no | no | yes | yes | |
| | | | colitis | | | | | | | | | |
| MICI149 | 26 | M | colitis | no | Active | 8 | yes | no | no | no | yes | anti-TNF |
| MICI150 | 59 | M | colitis | former | Quiescent | 1 | no | no | no | yes | yes | anti-TNF |
| MICI153 | 26 | M | colitis | yes | Active | 8 | no | no | no | intolerent | intolerent | anti-TNF |
| MICI156 | 47 | F | colitis | no | Quiescent | 21 | yes | no | no | no | yes | no |
| MICI157 | 56 | M | colitis | yes | Quiescent | 8 | yes | yes | no | no | no | |
| MICI158 | 36 | M | colitis | yes | Active | 10 | no | no | no | no | no | anti-TNF |

Patients with Ulcerative Colitis

| | Age | | | | | | | | | | | |
|---|---|---|---|---|---|---|---|---|---|---|---|---|
| | at | | | | | Disease | | | | | | |
| | biopsy | | Colitis | Cigarette | disease | duration | Cortico | 5asa | | Imurel | Imurel | other |
| Patient | (years) | Sex | grade | smoker | activity | (years) | dependance | treatment | Cholangitis | treatment | failure | treatments |
| MICI035 | 22 | M | E3 | no | Active | 1 | yes | no | no | no | | |
| MICI038 | 74 | F | E1 | no | Quiescent | 1 | no | yes | no | | | |
| MICI098 | 69 | M | | no | Quiescent | | no | | yes | | | |
| MICI101 | 38 | M | E3 | no | Quiescent | 10 | yes | yes | no | | yes | anti-TNF |
| MICI114 | 42 | M | E3 | no | Quiescent | 17 | no | no | yes | no | | |
| MICI115 | 68 | M | E3 | no | Active | 24 | no | yes | no | no | yes | metotrexate |
| MICI117 | 56 | M | E1 | no | Active | 9 | no | yes | no | no | | |
| MICI119 | 59 | M | E1 | no | Active | 9 | no | yes | no | no | | |
| MICI120 | 74 | F | E2 | former | Quiescent | 28 | no | yes | no | no | no | |
| MICI131 | 53 | F | E1 | no | Active | 9 | yes | yes | no | intolerent | intolerent | |
| MICI132 | 31 | F | E1 | former | Active | 4 | no | yes | no | yes | yes | |
| MICI133 | 57 | F | E1 | no | Quiescent | 1 | no | yes | no | | | |
| MICI134 | 32 | F | E1 | former | Active | 1 | no | yes | no | no | | |
| MICI135 | 25 | M | E2 | former | Active | 2 | yes | yes | no | yes | yes | |
| MICI136 | 60 | M | E2 | no | Active | 9 | yes | no | no | yes | yes | |
| MICI137 | 25 | F | E2 | no | Active | 5 | no | yes | no | yes | yes | anti-TNF |
| MICI138 | 25 | F | E2 | no | Quiescent | 7 | yes | yes | no | intolerent | intolerent | |
| MICI139 | 61 | F | E2 | no | Active | 7 | no | yes | no | no | | |
| MICI140 | 38 | M | E3 | former | Quiescent | 10 | no | no | yes | | | |
| MICI208 | | | | yes | Quiescent | | | | | | | |
| MICI209 | | | | yes | Quiescent | | | | | | | |
| MICI210 | | | | yes | Quiescent | | | | | | | |
| Non Inflammatory Bowel Disease Patients | | | | | | | | | | | | |
| | Age at | | | | | | | | | | | |
| | biopsy | | Cigarette | Disease | | | | | | | | |
| Patient | (years) | Sex | smokers | associated | | | | | | | | |
| MICI012 | 66 | F | no | normal | | | | | | | | |
| MICI048 | 44 | F | no | normal | | | | | | | | |
| MICI065 | 66 | M | no | polype | | | | | | | | |
| MICI129 | 65 | F | no | polype | | | | | | | | |
| MICI162 | 59 | M | no | normal | | | | | | | | |
| MICI164 | 70 | F | no | normal | | | | | | | | |
| MICI166 | 52 | M | no | normal | | | | | | | | |
| MICI168 | 34 | F | no | normal | | | | | | | | |
| MICI198 | | | yes | | | | | | | | | |
| MICI199 | | | yes | | | | | | | | | |
| MICI200 | | | yes | | | | | | | | | |
| MICI201 | | | yes | | | | | | | | | |
| MICI202 | | | yes | | | | | | | | | |
| MICI203 | | | yes | | | | | | | | | |
| MICI205 | | | yes | | | | | | | | | |
| MICI206 | | | yes | | | | | | | | | |
| MICI207 | | | yes | | | | | | | | | |

SUPPLEMENTARY TABLE 2
Expressions of human phase 1 and phase 2 metabolizing
enzymes, transporters and transcription factors mRNAs in ascending
colon of UC patients (vs expression in non IBD patients)

| | | |
|---|---|---|
| | Fold change | P |
| Gene Name | (M ± SEM) | value |

Phase 1 enzyme

| | | |
|---|---|---|
| ABP1 | 0.9682 ± 0.08713 | 0.449 |
| ADH1B-C | 1.094 ± 0.1403 | 0.500 |
| ADH4 | **0.288 ± 0.05245 | 0.009 |
| ADH5 | 1.224 ± 0.1339 | 0.230 |
| ADH6 | *1.226 ± 0.07713 | 0.035 |
| ADHFE1 | *0.5041 ± 0.06894 | 0.002 |
| AKR1A1 | *1.313 ± 0.09102 | 0.033 |
| AKR1B1 | 1.087 ± 0.1252 | 0.063 |
| AKR1B10 | 1.027 ± 0.1506 | 0.345 |
| AKR1C1-2 | 1.329 ± 0.2239 | 0.437 |
| AKR1C3 | 0.9018 ± 0.2229 | 0.187 |
| AKR1E2 | 0.9898 ± 0.2599 | 0.262 |
| AKR7A2 | *1.565 ± 0.1901 | 0.047 |
| AKR7A3 | 1.173 ± 0.1380 | 0.201 |
| ALDH16A1 | 1.271 ± 0.2192 | 0.437 |
| ALDH18A1 | 0.7878 ± 0.07793 | 0.163 |
| ALDH1A1 | 1.457 ± 0.1895 | 0.065 |
| ALDH1A3 | *0.5372 ± 0.07275 | 0.019 |
| ALDH1B1 | 1.099 ± 0.1073 | 0.345 |
| ALDH1L1 | *2.297 ± 0.3591 | 0.047 |
| ALDH2 | 1.186 ± 0.1100 | 0.230 |
| ALDH3A1 | 1.125 ± 0.1586 | 0.489 |
| ALDH3A2 | 1.13 ± 0.09181 | 0.213 |
| ALDH3B1 | 0.6141 ± 0.06958 | 0.132 |
| ALDH4A1 | 1.381 ± 1.381 | 0.077 |
| ALDH5A1 | 0.9665 ± 0.05845 | 0.437 |
| ALDH6A1 | 1.085 ± 0.08048 | 0.316 |
| ALDH7A1 | *1.455 ± 0.1487 | 0.030 |
| ALDH9A1 | 1.071 ± 0.05198 | 0.333 |
| AOC3 | 1.047 ± 0.2720 | 0.191 |
| AOX1 | *0.5147 ± 0.1564 | 0.023 |
| BCHE | *0.6255 ± 0.1148 | 0.038 |
| CBR1 | 1.249 ± 0.1691 | 0.230 |
| CBR3 | *1.698 ± 0.2201 | 0.026 |
| CBR4 | 0.8543 ± 0.09840 | 0.167 |
| CES1 | **2.695 ± 0.5435 | 0.008 |
| CES2 | 1.004 ± 0.09929 | 0.449 |
| CES3 | 0.8369 ± 0.06544 | 0.289 |
| CYP1B1 | *0.3006 ± 0.03749 | 0.042 |
| CYP26B1 | 0.6786 ± 0.1291 | 0.288 |
| CYP27A1 | 1.08 ± 0.1502 | 0.406 |
| CYP2B6 | 1.424 ± 0.2643 | 0.297 |
| CYP2C8-19 | 0.9948 ± 0.1675 | 0.365 |
| CYP2E1 | *0.415 ± 0.1056 | 0.044 |
| CYP2J2 | 0.995 ± 0.06220 | 0.395 |
| CYP2R1 | 0.7325 ± 0.09368 | 0.085 |
| CYP2S1 | 0.9072 ± 0.1780 | 0.385 |
| CYP2U1 | 0.6903 ± 0.07955 | 0.452 |
| CYP2W1 | *0.1518 ± 0.08839 | 0.032 |
| CYP20A1 | 1.008 ± 0.05918 | 0.468 |
| CYP27B1 | 1.097 ± 0.08893 | 0.271 |
| CYP3A5 | 0.6965 ± 0.1697 | 0.155 |
| CYP4F11 | *0.5877 ± 0.09763 | 0.017 |
| CYP4F12 | 0.8755 ± 0.1256 | 0.238 |
| CYP4F2 | 0.6425 ± 0.08084 | 0.479 |
| CYP4V2 | 0.8502 ± 0.07018 | 0.121 |
| CYP4X1 | 1.431 ± 0.2398 | 0.161 |
| CYP51A1 | *1.486 ± 0.1171 | 0.016 |
| DHRS4 | 1.216 ± 0.1027 | 0.097 |
| DHRS9 | 1.092 ± 0.1176 | 0.371 |
| DPYD | 0.8617 ± 0.1344 | 0.176 |
| EPHX1 | 1.211 ± 0.1586 | 0.297 |
| EPHX2 | 1.058 ± 0.1091 | 0.370 |
| ESD | *1.368 ± 0.1153 | 0.032 |
| FMO4 | 0.7802 ± 0.09783 | 0.333 |
| FMO5 | 0.712 ± 0.08074 | 0.052 |
| HSD17B10 | 1.127 ± 0.07797 | 0.127 |
| KCNAB2 | *1.267 ± 0.09129 | 0.012 |
| KDM1A | 1.185 ± 0.08734 | 0.111 |
| KDM1B | 0.9171 ± 0.09764 | 0.333 |
| MAOA | 1.005 ± 0.1132 | 0.449 |
| MAOB | 0.7557 ± 0.09749 | 0.097 |
| NQO1 | 1.387 ± 0.2055 | 0.161 |
| NQO2 | 1.171 ± 0.1307 | 0.238 |
| PAOX | 1.202 ± 0.09565 | 0.137 |
| PON2 | 0.9521 ± 0.09617 | 0.307 |
| PON3 | 1.242 ± 0.1544 | 0.163 |
| PTGIS | 0.4627 ± 0.1261 | 0.111 |
| SPR | 1.017 ± 0.1165 | 0.489 |
| SUOX | 0.8899 ± 0.08739 | 0.245 |
| XDH | 1.159 ± 0.2094 | 0.336 |

Phase 2 enzyme

| | | |
|---|---|---|
| AS3MT | 1.197 ± 0.1269 | 0.215 |
| COMT | **1.506 ± 0.1058 | 0.008 |
| GGT1 | 0.8268 ± 0.1280 | 0.262 |
| GSTA1 | 2.364 ± 0.5976 | 0.080 |
| GSTA4 | *1.438 ± 0.1775 | 0.047 |
| GSTK1 | 1.152 ± 0.1112 | 0.237 |
| GSTM2 | 0.9053 ± 0.07345 | 0.345 |
| GSTM3 | 1.362 ± 0.1586 | 0.137 |
| GSTM4 | 1.304 ± 0.1144 | 0.081 |
| GSTM5 | 1.144 ± 0.1421 | 0.380 |
| GSTO1 | 0.8992 ± 0.1977 | 0.205 |
| GSTP1 | **1.673 ± 0.1160 | 0.009 |
| GSTT1 | 1.373 ± 0.2472 | 0.297 |
| GSTZ1 | 1.391 ± 0.1217 | 0.074 |
| HNMT | 0.9413 ± 0.1169 | 0.395 |
| INMT | **0.5359 ± 0.1033 | 0.008 |
| MGST1 | 1.245 ± 0.1079 | 0.106 |
| MGST2 | *1.356 ± 0.09061 | 0.038 |
| MGST3 | 1.304 ± 0.1202 | 0.080 |
| NAT1 | 1.091 ± 0.09207 | 0.307 |
| NAT2 | 2.089 ± 0.6124 | 0.087 |
| NAA20 | 1.122 ± 0.06335 | 0.072 |
| NNMT | 1.928 ± 0.4130 | 0.081 |
| SULT1A2 | 0.7278 ± 0.09846 | 0.395 |
| SULT1A3/4 | 0.961 ± 0.1173 | 0.447 |
| SULT1B1 | 1.516 ± 0.4439 | 0.380 |
| SULT1C2 | 1.502 ± 0.5555 | 0.315 |
| SULT1C4 | 1.538 ± 0.2525 | 0.144 |
| SULT2A1 | **0.05938 ± 0.02384 | 0.001 |
| SULT2B1 | 1.738 ± 0.3398 | 0.205 |
| TST | 1.135 ± 0.1316 | 0.298 |
| TPMT | *0.6398 ± 0.08109 | 0.042 |
| UGT1A1 | 1.354 ± 0.4190 | 0.355 |
| UGT1A10 | 1.019 ± 0.1651 | 0.470 |
| UGT1A4 | **0.147 ± 0.07438 | 0.005 |
| UGT1A5 | 0.8015 ± 0.1892 | 0.266 |
| UGT1A6 | 1.27 ± 0.1785 | 0.194 |
| UGT1A7 | 0.6485 ± 0.1539 | 0.134 |
| UGT1A8 | 1.057 ± 0.2331 | 0.419 |
| UGT1A9 | *0.6331 ± 0.08243 | 0.042 |
| UGT2A3 | 0.7093 ± 0.08501 | 0.088 |
| UGT2B10 | 0.9731 ± 0.4438 | 0.087 |
| UGT2B11 | 0.4272 ± 0.1774 | 0.326 |
| UGT2B17 | 1.206 ± 0.5476 | 0.423 |
| UGT2B7 | *0.4597 ± 0.05120 | 0.019 |
| UGT8 | 0.941 ± 0.07315 | 0.390 |

Transporters

| | | |
|---|---|---|
| ABCA1 | **0.5064 ± 0.06142 | 0.001 |
| ABCA2 | **0.4707 ± 0.07822 | 0.002 |
| ABCA3 | 0.6475 ± 0.1664 | 0.149 |
| ABCA8 | 0.8578 ± 0.1178 | 0.149 |
| ABCB1 | *0.6644 ± 0.08195 | 0.026 |
| ABCB10 | 0.8496 ± 0.07623 | 0.144 |
| ABCB11 | 0.6441 ± 0.1397 | 0.065 |
| ABCB4 | 0.5025 ± 0.08290 | 0.074 |
| ABCB6 | 1.257 ± 0.1307 | 0.132 |
| ABCB7 | 1.035 ± 0.07350 | 0.365 |
| ABCB8 | 0.9019 ± 0.07386 | 0.157 |
| ABCB9 | 0.8568 ± 0.07050 | 0.221 |
| ABCC1 | *1.324 ± 0.1083 | 0.042 |
| ABCC10 | *0.7569 ± 0.05399 | 0.025 |
| ABCC3 | 0.8827 ± 0.08621 | 0.201 |
| ABCC4 | 0.8112 ± 0.08440 | 0.449 |
| ABCC5 | **0.6364 ± 0.07989 | 0.009 |
| ABCC6 | **0.6304 ± 0.08677 | 0.007 |
| ABCD4 | 1.047 ± 0.07830 | 0.163 |
| ABCG2 | *0.6795 ± 0.1549 | 0.042 |
| AQP1 | 1.355 ± 0.2316 | 0.191 |
| ATP6V0C | 1.281 ± 0.1759 | 0.176 |
| ATP7A | **0.5588 ± 0.06695 | 0.008 |
| ATP7B | 1.074 ± 0.07617 | 0.280 |
| MVP | 1.02 ± 0.1386 | 0.385 |
| SLC1A1 | 1.112 ± 0.1003 | 0.315 |
| SLC1A3 | *0.3145 ± 0.06901 | 0.022 |
| SLC2A1 | 1.035 ± 0.1161 | 0.490 |
| SLC3A1 | 0.6833 ± 0.1048 | 0.053 |
| SLC3A2 | 0.8847 ± 0.06334 | 0.406 |
| SLC6A4 | 0.7632 ± 0.1787 | 0.280 |
| SLC7A5 | **0.5477 ± 0.1037 | 0.004 |
| SLC7A7 | 1.33 ± 0.1789 | 0.209 |
| SLC7A6 | 0.7431 ± 0.06694 | 0.053 |
| SLC7A8 | 1.287 ± 0.1016 | 0.077 |
| SLC7A11 | 1.24 ± 0.2154 | 0.213 |
| SLC10A2 | **0.2868 ± 0.08512 | 0.008 |
| SLC15A1 | *0.5608 ± 0.1357 | 0.041 |
| SLC15A2 | **0.2637 ± 0.03431 | 0.005 |
| SLC16A1 | 0.7546 ± 0.1323 | 0.127 |
| SLC18A2 | 0.8418 ± 0.3335 | 0.262 |
| SLC19A1 | 0.8544 ± 0.2816 | 0.079 |
| SLC19A2 | **0.5761 ± 0.05615 | 0.002 |
| SLC19A3 | *0.7068 ± 0.09741 | 0.049 |
| SLC22A3 | *0.6432 ± 0.08283 | 0.028 |
| SLC22A4 | 0.9377 ± 0.1509 | 0.142 |
| SLC22A5 | 0.7458 ± 0.09685 | 0.080 |
| SLC25A13 | 1.002 ± 0.09206 | 0.500 |
| SLC28A2 | 1.683 ± 0.4095 | 0.253 |
| SLC28A3 | *2.234 ± 0.4034 | 0.026 |
| SLC29A1 | 1.162 ± 0.1101 | 0.271 |
| SLC29A2 | *1.566 ± 0.1725 | 0.013 |
| SLC29A3 | 1.339 ± 0.2384 | 0.443 |
| SLC29A4 | 1.606 ± 0.4687 | 0.472 |
| SLC31A1 | 1.11 ± 0.06407 | 0.213 |
| SLC38A1 | *2.925 ± 1.008 | 0.040 |
| SLC38A2 | 0.8972 ± 0.07526 | 0.089 |
| SLC38A5 | **1.603 ± 0.1525 | 0.009 |
| SLC47A1 | **0.1672 ± 0.07138 | 0.005 |
| SLCO2A1 | 1.102 ± 0.1686 | 0.419 |
| SLCO2B1 | *0.6603 ± 0.09789 | 0.022 |
| SLCO3A1 | 0.9147 ± 0.1664 | 0.326 |
| SLCO4A1 | 1.074 ± 0.1190 | 0.176 |
| SLCO4C1 | *0.3676 ± 0.08220 | 0.012 |
| TAP1 | 1.077 ± 0.1343 | 0.230 |
| TAP2 | 0.802 ± 0.1224 | 0.097 |
| VDAC2 | 1.296 ± 0.1679 | 0.161 |
| VDAC3 | 1.07 ± 0.05258 | 0.500 |

Nuclear receptors & transcription factors

| | | |
|---|---|---|
| AIP | 0.8669 ± 0.06061 | 0.194 |
| AHR | 1.112 ± 0.1003 | 0.315 |
| ARNT | **0.7748 ± 0.04807 | 0.006 |
| CREBBP | 1.19 ± 0.1789 | 0.298 |
| EP300 | 0.8334 ± 0.08502 | 0.126 |
| FOXA2 | 0.7359 ± 0.1320 | 0.126 |
| FOXO1 | *0.594 ± 0.09479 | 0.021 |
| HIF1A | 0.9335 ± 0.08654 | 0.271 |
| HIF3A | **0.08911 ± 0.03896 | 0.001 |
| HNF4A | 0.8343 ± 0.1150 | 0.116 |
| HSP90AA1 | 1.153 ± 0.1398 | 0.395 |
| KEAP1 | 1.219 ± 0.09947 | 0.054 |
| NCOA1 | 0.9534 ± 0.07408 | 0.385 |
| NCOA2 | *0.6189 ± 0.03901 | 0.014 |
| NCOA3 | 0.811 ± 0.07964 | 0.089 |
| NCOR1 | 0.9824 ± 0.08916 | 0.263 |
| NCOR2 | *0.4617 ± 0.09711 | 0.027 |
| NR0B2 | 0.5828 ± 0.1152 | 0.067 |
| NR1H2 | 1.148 ± 0.1813 | 0.410 |
| NR1H3 | *0.5503 ± 0.06815 | 0.047 |
| NR1H4 | 0.7458 ± 0.1530 | 0.215 |
| NR112 | 1.173 ± 0.2987 | 0.126 |
| NR113 (CAR) | 1.909 ± 0.6455 | 0.370 |
| NR3C1 | *0.6019 ± 0.06750 | 0.017 |
| NR3C2 | 0.8409 ± 0.07607 | 0.167 |
| NR5A2 | 0.7546 ± 0.1121 | 0.056 |
| NRF2 | 0.9921 ± 0.07616 | 0.449 |
| PPARA | 0.7001 ± 0.1068 | 0.067 |
| PPARD | **0.4434 ± 0.07243 | 0.006 |
| PPARG | 1.026 ± 0.2612 | 0.106 |
| PPARGC1A | *0.7834 ± 0.07474 | 0.025 |
| PPARGC1B | 1.196 ± 0.1469 | 0.355 |
| PPRC1 | 1.305 ± 0.1424 | 0.093 |
| PTGES3 | 1.116 ± 0.1697 | 0.352 |
| RARA | 0.89 ± 0.1219 | 0.187 |
| RARB | **0.329 ± 0.06700 | 0.002 |
| RARG | 1.067 ± 0.07574 | 0.400 |
| RXRA | 0.6287 ± 0.08401 | 0.059 |
| RXRB | *0.6902 ± 0.09052 | 0.042 |
| THRA | 1.153 ± 0.1040 | 0.297 |
| THRB | **0.5036 ± 0.06258 | 0.002 |
| TRIP11 | 1.08 ± 0.1107 | 0.470 |
| VDR | 1.016 ± 0.1586 | 0.298 |

Other genes

| | | |
|---|---|---|
| CRABP1 | 0.665 ± 0.1901 | 0.097 |
| MTHFR | 0.8695 ± 0.07243 | 0.106 |
| CRABP2 | 0.7025 ± 0.1166 | 0.161 |
| GZMA | 1.29 ± 0.1671 | 0.183 |
| POR | 0.9978 ± 0.1330 | 0.263 |
| LW | 1.071 ± 0.08074 | 0.306 |

SUPPLEMENTARY TABLE 4
Expressions of 65 human phase 1 and phase 2 metabolizing
enzymes, transporters and transcription factors mRNAs which were showed deregulated in
ascending colon of UC patients in 9 smoking controls, 19 non-smokingUC patients and 3
smoking UC patients.

| | | | | | Smoking UC |
|---|---|---|---|---|---|
| | Smoking Controls | | Non-smoking UC patients | | patients |
| | Fold change | | Fold change | P | Fold change |
| Gene Name | (M ± SEM) | P value | (M ± SEM) | value | (M ± SEM) |

Phase 1 enzyme

| | | | | | |
|---|---|---|---|---|---|
| ADH4 | 2.202 ± 0.56 | 0.1135 | **0.288 ± 0.052 | 0.009 | 1.231 ± 0.366 |
| ADH6 | 0.729 ± 0.098 | 0.0567 | *1.226 ± 0.077 | 0.035 | 0.11 ± 0.031 |
| ADHFE1 | **1.96 ± 0.299 | 0.0059 | *0.504 ± 0.069 | 0.002 | 0.967 ± 0.179 |
| AKR1A1 | 0.879 ± 0.065 | 0.1001 | *1.313 ± 0.091 | 0.033 | 2.198 ± 1.135 |
| AKR7A2 | 0.928 ± 0.148 | 0.3332 | *1.565 ± 0.19 | 0.047 | 1.771 ± 0.72 |
| ALDH1A3 | *3.111 ± 0.723 | 0.02 | *0.537 ± 0.073 | 0.019 | 0.859 ± 0.141 |
| ALDH1L1 | 0.941 ± 0.163 | 0.4317 | *2.297 ± 0.359 | 0.047 | 3.247 ± 0.289 |
| ALDH7A1 | *0.649 ± 0.099 | 0.0288 | *1.455 ± 0.149 | 0.030 | 1.712 ± 0.104 |
| AOX1 | *3.162 ± 0.709 | 0.0122 | *0.515 ± 0.156 | 0.023 | 2.796 ± 1.776 |
| BCHE | 1.562 ± 0.281 | 0.0567 | *0.626 ± 0.115 | 0.038 | 0.448 ± 0.163 |
| CBR3 | 0.94 ± 0.097 | 0.4657 | *1.698 ± 0.22 | 0.026 | 0.816 ± 0.313 |
| CES1 | *1.692 ± 0.397 | 0.0385 | **2.695 ± 0.544 | 0.008 | 5.86 ± 2.14 |
| CYP1B1 | ***16.12 ± 3.654 | <0.0001 | *0.301 ± 0.037 | 0.042 | 3.334 ± 1.492 |
| CYP2E1 | **3.124 ± 0.68 | 0.0053 | *0.415 ± 0.106 | 0.044 | 1.925 ± 1.448 |
| CYP2W1 | ***119.7 ± 30.67 | <0.0001 | *0.152 ± 0.088 | 0.032 | 3.197 ± 0.944 |
| CYP4F11 | 2.2 ± 0.506 | 0.068 | *0.588 ± 0.098 | 0.017 | 1.517 ± 0.577 |
| CYP51A1 | ***2.419 ± 0.325 | 0.001 | *1.486 ± 0.117 | 0.016 | 2.637 ± 1.03 |
| ESD | *0.573 ± 0.08 | 0.0157 | *1.368 ± 0.115 | 0.032 | 1.213 ± 0.129 |
| KCNAB2 | 1.052 ± 0.394 | 0.1487 | *1.267 ± 0.0913 | 0.012 | 0.027 ± 0.017 |

Phase 2 enzyme

| | | | | | |
|---|---|---|---|---|---|
| COMT | 0.805 ± 0.1021 | 0.193 | **1.506 ± 0.106 | 0.008 | 1.746 ± 0.342 |
| GSTA4 | **0.475 ± 0.071 | 0.004 | *1.438 ± 0.178 | 0.047 | 1.725 ± 0.464 |
| GSTP1 | 0.56 ± 0.10413 | 0.057 | **1.673 ± 0.116 | 0.009 | 1.285 ± 0.376 |
| INMT | *2.351 ± 0.539 | 0.025 | **0.5359 ± 0.103 | 0.008 | 0.864 ± 0.288 |
| MGST2 | 0.772 ± 0.086 | 0.057 | *1.356 ± 0.091 | 0.038 | 1.41 ± 0.185 |
| SULT2A1 | **1.25 ± 0.267 | 0.002 | **0.06 ± 0.024 | 0.001 | 1.568 ± 1.011 |
| TPMT | *1.762 ± 0.16 | 0.012 | *0.64 ± 0.081 | 0.042 | 1.047 ± 0.208 |
| UGT1A4 | 0.554 ± 0.094 | 0.129 | **0.147 ± 0.074 | 0.005 | 2.488 ± 0.698 |
| UGT1A9 | *1.917 ± 0.362 | 0.016 | *0.633 ± 0.082 | 0.042 | 0.425 ± 0.214 |
| UGT2B7 | 0.86 ± 0.158 | 0.333 | *0.46 ± 0.051 | 0.019 | 2.081 ± 1.486 |

Transporters

| | | | | | |
|---|---|---|---|---|---|
| ABCA1 | 0.969 ± 0.19 | 0.333 | **0.506 ± 0.061 | 0.001 | 0.461 ± 0.27 |
| ABCA2 | 0.671 ± 0.091 | 0.193 | **0.471 ± 0.078 | 0.002 | 3.819 ± 3.705 |
| ABCB1 | 1.394 ± 0.26 | 0.193 | *0.664 ± 0.082 | 0.026 | 0.396 ± 0.224 |
| ABCC1 | *1.288 ± 0.155 | 0.047 | *1.324 ± 0.108 | 0.042 | 1.488 ± 0.448 |
| ABCC10 | *0.543 ± 0.112 | 0.014 | *0.757 ± 0.054 | 0.025 | 0.953 ± 0.504 |
| ABCC5 | 1.975 ± 0.421 | 0.068 | **0.636 ± 0.08 | 0.009 | 0.659 ± 0.07 |
| ABCC6 | *0.516 ± 0.103 | 0.020 | **0.63 ± 0.087 | 0.007 | 0.967 ± 0.739 |
| ABCG2 | 0.967 ± 0.18 | 0.365 | *0.68 ± 0.155 | 0.042 | 0.517 ± 0.136 |
| ATP7A | 1.471 ± 0.293 | 0.125 | **0.559 ± 0.067 | 0.008 | 2.601 ± 0.873 |
| SLC1A3 | 1.195 ± 0.236 | 0.302 | *0.315 ± 0.069 | 0.022 | 2.171 ± 1.903 |
| SLC7A5 | 0.942 ± 0.151 | 0.372 | **0.548 ± 0.104 | 0.004 | 5.631 ± 5.539 |
| SLC10A2 | 1.947 ± 0.486 | 0.068 | **0.287 ± 0.085 | 0.008 | 28.14 ± 27.76 |
| SLC15A1 | 1.966 ± 0.742 | 0.213 | *0.561 ± 0.136 | 0.041 | 0.416 ± 0.294 |
| SLC15A2 | ***6.395 ± 1.168 | <0.0001 | **0.264 ± 0.034 | 0.005 | 0.929 ± 0.485 |
| SLC19A2 | 1.266 ± 0.154 | 0.149 | **0.576 ± 0.056 | 0.002 | 1.039 ± 0.559 |
| SLC19A3 | 1.355 ± 0.2067 | 0.111 | *0.707 ± 0.097 | 0.049 | 1.947 ± 0.72 |
| SLC22A3 | 0.838 ± 0.132 | 0.430 | *0.643 ± 0.083 | 0.028 | 0.923 ± 0.263 |
| SLC28A3 | 0.924 ± 0.172 | 0.081 | *2.234 ± 0.403 | 0.026 | 1.748 ± 1.196 |
| SLC29A2 | 0.918 ± 0.154 | 0.302 | *1.566 ± 0.173 | 0.013 | 0.939 ± 0.419 |
| SLC38A1 | 0.978 ± 0.114 | 0.465 | *2.925 ± 1.008 | 0.040 | 0.678 ± 0.246 |
| SLC38A5 | 0.725 ± 0.141 | 0.245 | **1.603 ± 0.153 | 0.009 | 1.134 ± 0.139 |
| SLC47A1 | ***11.33 ± 2.795 | 0.001 | **0.167 ± 0.071 | 0.005 | 2.589 ± 0.699 |
| SLCO2B1 | *0.589 ± 0.085 | 0.016 | *0.66 ± 0.098 | 0.022 | 0.848 ± 0.155 |
| SLCO4C1 | ***4.156 ± 0.703 | 0.0002 | *0.368 ± 0.082 | 0.012 | 2.101 ± 0.484 |

Nuclear receptors & transcription factors

| | | | | | |
|---|---|---|---|---|---|
| ARNT | 1.178 ± 0.129 | 0.165 | **0.775 ± 0.048 | 0.006 | 1.001 ± 0.241 |
| FOXO1 | 0.966 ± 0.133 | 0.482 | *0.594 ± 0.095 | 0.021 | 0.411 ± 0.098 |
| HIF3A | **4.88 ± 1.478 | 0.004 | **0.089 ± 0.039 | 0.001 | 1.723 ± 0.944 |
| NCOA2 | ***3.324 ± 0.576 | <0.0001 | *0.619 ± 0.039 | 0.014 | 2.111 ± 0.633 |
| NCOR2 | 0.881 ± 0.085 | 0.3652 | *0.462 ± 0.097 | 0.027 | 1.641 ± 1.217 |
| NR1H3 | **0.516 ± 0.063 | 0.006 | *0.55 ± 0.068 | 0.047 | 0.865 ± 0.273 |
| NR3C1 | 0.839 ± 0.121 | 0.333 | *0.602 ± 0.068 | 0.017 | 0.759 ± 0.303 |
| PPARD | ***3.53 ± 0.575 | 0.0001 | **0.443 ± 0.072 | 0.006 | 2.362 ± 0.585 |
| PPARGC1A | ***1.844 ± 0.212 | 0.0004 | *0.783 ± 0.075 | 0.025 | 1.164 ± 0.306 |
| RARB | **3.215 ± 0.805 | 0.004 | **0.329 ± 0.067 | 0.002 | 2.698 ± 1.208 |
| RXRB | 0.861 ± 0.099 | 0.226 | *0.69 ± 0.091 | 0.042 | 1.438 ± 0.329 |
| THRB | 1.259 ± 0.215 | 0.170 | **0.504 ± 0.063 | 0.002 | 0.145 ± 0.021 |

### REFERENCES

Throughout this application, various references describe the state of the art to which this invention pertains. The disclosures of these references are hereby incorporated by reference into the present disclosure.
1 Mahid S S, et al. Mayo Clin Proc 2006; 81:1462-71.
2 Nakata K et al. Drug Metab Pharmacokinet 2006; 21:437-57.
3 Bourgine J, et al. Drug Metab Dispos 2012; 40:694-705.
4 Roediger W E, et al. Gut 1997; 41:731-4.
5 Monteleone I, et al. Gastroenterology 2012; 141:237-48, 48 el.
6 Panwala C M, et al. J Immunol 1998; 161:5733-44.
7 Treton X, et al. Gastroenterology 2011; 141:1024-35.
8 Deuring J J, et al. Biochem J 2012; 441:87-93.
9 Langmann T, et al. Gastroenterology 2004; 127:26-40.
10 Schwab M, et al. Gastroenterology 2003; 124:26-33.
11 Furumatsu K, et al. Dig Dis Sci 2011; 56:2532-44.
12 Bertin B, et al. Curr Drug Targets 2013.
13 Heimerl S, et al. Biochim Biophys Acta 2006; 1762:341-50.

1. A method for diagnosing ulcerative colitis in a subject comprising the steps of i) determining in a sample obtained from the subject an expression level of at least one gene selected from the group consisting of ADH4, ADH6, ADHFE1, AKR1A1, AKR7A2, ALDH1A3, ALDH1L1, ALDH7A1, AOX1, BCHE, CBR3, CES1, CYP1B1, CYP2E1, CYP2W1, CYP4F11, CYP51A1, ESD, KCNAB2, COMT, GSTA4, GSTP1, INMT, MGST2, SULT2A1, TPMT, UGT1A4, UGT1A9, UGT2B7, ABCA1, ABCA2, ABCB1, ABCC1, ABCC10, ABCC5, ABCC6, ABCG2, ATP7A, SLC1A3, SLC7A5, SLC10A2, SLC15A1, SLC15A2, SLC19A2, SLC19A3, SLC22A3, SLC28A3, SLC29A2, SLC38A1, SLC38A5, SLC47A1, SLCO2B1, SLCO4C1, ARNT, FOXO1, HIF3A, NCOA2, NCOR2, NR1H3, NR3C1, PPARD, PPARGC1A, RARB, RXRB, and THRB, ii) comparing the expression level determined at step i) with a reference value and iii) concluding that the subject suffers from ulcerative colitis when one or both of the following are found:
the expression determined at step i) is higher than the reference value for at least one gene selected from the group consisting of ADH6, AKR1A1, AKR7A2, ALDH1L1, ALDH7A1, CBR3, CES1, CYP51A1, ESD, KCNAB2, COMT, GSTA4, GSTP1, MGST2, UGT2B7, ABCC1, SLC28A3, SLC29A2, SLC38A1, and SLC38A5, and
the expression determined at step at step i) is lower than the reference value for at least one gene selected from the group consisting of ADH4, ADHFE1, ALDH1A3, AOX1, BCHE, CYP1B1, CYP2E1, CYP2W1, CYP4F11, INMT, SULT2A1, TPMT, UGT1A4, UGT1A9, ABCA1, ABCA2, ABCB1, ABCC10, ABCC5, ABCC6, ABCG2, ATP7A, SLC1A3, SLC7A5, SLC10A2, SLC15A1, SLC15A2, SLC19A2, SLC19A3, SLC22A3, SLC47A1, SLCO2B1, SLCO4C1, ARNT, FOXO1, HIF3A, NCOA2, NCOR2, NR1H3, NR3C1, PPARD, PPARGC1A, RARB, RXRB, and THRB.

2. The method of claim 1 wherein the expression level of 1; 2; 3; 4; 5; 6; 7; 8; 9; 10; 11; 12; 13; 14; 15; 16; 17; 18; 19; 20; 21; 22; 23; 24; 25; 26; 27; 28; 29; 30; 31; 32; 33; 34; 35; 36; 37; 38; 39; 40; 41; 42; 43; 44; 45; 46; 47; 48; 49; 50; 51; 52; 53; 54; 55; 56; 57; 58; 59; 60; 61; 62; 63; 64 or 65 genes is determined.

3. The method of claim 1 which further comprises a step of i) determining an expression level of at least one miRNA selected from the group consisting of miR15a, miR26a, miR29a, miR29b, miR30c, miR126*, miR127-3p, miR-142-3p, miR-142-5p, miR-146a, miR-146b-5p, miR150, miR-181d, miR-182, miR185, miR196a, miR199a-3p, miR199a-5p, miR199b-5p, miR-203, miR223, miR-299-5p, miR320a, miR324-3p, and miR-328, ii) comparing the expression level determined at step i) with a reference value and iii) concluding that the subject suffers from an ulcerative disease when one or both of the following are found:
the expression determined at step i) is higher than the reference value for at least one miRNA selected from the group consisting of miR15a, miR26a, miR29a, miR29b, miR30c, miR126*, miR127-3p, miR185, miR196a, miR324-3p, and miR-146b-5p and
the expression determined at step at step i) is lower than the reference value for at least one miRNA selected from the group consisting of miR150, miR-181d, miR-182, miR199a-3p, miR199a-5p, miR199b-5p, miR-203, miR223, miR-299-5p, miR320a, miR-146a, miR-142-3p, miR-142-5p, and miR-328.

4. The method of claim 3 wherein the expression level of 1; 2; 3; 4; 5; 6; 7; 8; 9; 10; 11; 12; 13; 14; 15; 16; 17; 18; 19; 20; 21; 22; 23; 24; or 25 miRNA(s) is determined.

5. A chip comprising a solid support which carries at least one nucleic acid specific for detecting at least one gene selected from the group consisting of ADH4, ADH6, ADHFE1, AKR1A1, AKR7A2, ALDH1A3, ALDH1L1, ALDH7A1, AOX1, BCHE, CBR3, CES1, CYP1B1, CYP2E1, CYP2W1, CYP4F11, CYP51A1, ESD, KCNAB2, COMT, GSTA4, GSTP1, INMT, MGST2, SULT2A1, TPMT, UGT1A4, UGT1A9, UGT2B7, ABCA1, ABCA2, ABCB1, ABCC1, ABCC10, ABCC5, ABCC6, ABCG2, ATP7A, SLC1A3, SLC7A5, SLC10A2, SLC15A1, SLC15A2, SLC19A2, SLC19A3, SLC22A3, SLC28A3, SLC29A2, SLC38A1, SLC38A5, SLC47A1, SLCO2B1, SLCO4C1, ARNT, FOXO1, HIF3A, NCOA2, NCOR2, NR1H3, NR3C1, PPARD, PPARGC1A, RARB, RXRB, and THRB.

6. The chip of claim 5 wherein the solid support of the chip carries a set of nucleic acids specific for detecting 2; 3; 4; 5; 6; 7; 8; 9; 10; 11; 12; 13; 14; 15; 16; 17; 18; 19; 20; 21; 22; 23; 24; 25; 26; 27; 28; 29; 30; 31; 32; 33; 34; 35; 36; 37; 38; 39; 40; 41; 42; 43; 44; 45; 46; 47; 48; 49; 50; 51; 52; 53; 54; 55; 56; 57; 58; 59; 60; 61; 62; 63; 64 or 65 genes.

7. The chip of claim 5 wherein the solid support of the chip carries at least one specific nucleic acid specific for detecting at least one miRNA selected from the group consisting of miR15a, miR26a, miR29a, miR29b, miR30c, miR126*, miR127-3p, miR-142-3p, miR-142-5p, miR-146a, miR-146b-5p, miR150, miR-181d, miR-182, miR185, miR196a, miR199a-3p, miR199a-5p, miR199b-5p, miR-203, miR223, miR-299-5p, miR320a, miR324-3p, and miR-328.

8. The chip of claim 7 wherein the solid support of the chip carries a set of nucleic acids specific for detecting 1; 2; 3; 4; 5; 6; 7; 8; 9; 10; 11; 12; 13; 14; 15; 16; 17; 18; 19; 20; 21; 22; 23; 24; 25 miRNA(s).

9. A kit comprising means for determining the expression level of at least one gene selected from the group consisting of ADH4, ADH6, ADHFE1, AKR1A1, AKR7A2, ALDH1A3, ALDH1L1, ALDH7A1, AOX1, BCHE, CBR3, CES1, CYP1B1, CYP2E1, CYP2W1, CYP4F11, CYP51A1, ESD, KCNAB2, COMT, GSTA4, GSTP1, INMT, MGST2, SULT2A1, TPMT, UGT1A4, UGT1A9, UGT2B7, ABCA1, ABCA2, ABCB1, ABCC1, ABCC10, ABCC5, ABCC6, ABCG2, ATP7A, SLC1A3, SLC7A5, SLC10A2, SLC15A1, SLC15A2, SLC19A2, SLC19A3, SLC22A3, SLC28A3, SLC29A2, SLC38A1, SLC38A5, SLC47A1, SLCO2B1, SLCO4C1, ARNT, FOXO1, HIF3A, NCOA2, NCOR2, NR1H3, NR3C1, PPARD, PPARGC1A, RARB, RXRB, and THRB.

10. The kit of claim 9 which comprises means for determining the expression of 1; 2; 3; 4; 5; 6; 7; 8; 9; 10; 11; 12; 13; 14; 15; 16; 17; 18; 19; 20; 21; 22; 23; 24; 25; 26; 27; 28; 29; 30; 31; 32; 33; 34; 35; 36; 37; 38; 39; 40; 41; 42; 43; 44; 45; 46; 47; 48; 49; 50; 51; 52; 53; 54; 55; 56; 57; 58; 59; 60; 61; 62; 63; 64 or 65 genes.

11. The kit of claim 9 which further comprises means for determining the level of least one miRNA selected from the group consisting of miR15a, miR26a, miR29a, miR29b, miR30c, miR126*, miR127-3p, miR-142-3p, miR-142-5p, miR-146a, miR-146b-5p, miR150, miR-181d, miR-182, miR185, miR196a, miR199a-3p, miR199a-5p, miR199b-5p, miR-203, miR223, miR-299-5p, miR320a, miR324-3p, and miR-328.

12. The kit of claim 11 which comprises means for determining the expression level of 1; 2; 3; 4; 5; 6; 7; 8; 9; 10; 11; 12; 13; 14; 15; 16; 17; 18; 19; 20; 21; 22; 23; 24; 25 miRNA(s).

13. A method for diagnosing ulcerative colitis in a subject comprising the steps of
i) conducting a polymerase chain reaction (PCR), comprising
extracting total mRNA from a colonic sample;
converting the mRNA to cDNA;
amplifying cDNA corresponding to at least one gene selected from the group consisting of ADH4, ADH6, ADHFE1, AKR1A1, AKR7A2, ALDH1A3, ALDH1L1, ALDH7A1, AOX1, BCHE, CBR3, CES1, CYP1B1, CYP2E1, CYP2W1, CYP4F11, CYP51A1, ESD, KCNAB2, COMT, GSTA4, GSTP1, INMT, MGST2, SULT2A1, TPMT, UGT1A4, UGT1A9, UGT2B7, ABCA1, ABCA2, ABCB1, ABCC1, ABCC10, ABCC5, ABCC6, ABCG2, ATP7A, SLC1A3, SLC7A5, SLC10A2, SLC15A1, SLC15A2, SLC19A2, SLC19A3, SLC22A3, SLC28A3, SLC29A2, SLC38A1, SLC38A5, SLC47A1, SLCO2B1, SLCO4C1, ARNT, FOXO1, HIF3A, NCOA2, NCOR2, NR1H3, NR3C1, PPARD, PPARGC1A, RARB, RXRB, and THRB, and
ii) detecting amplification products.

14. The method of claim 13, wherein the step of amplifying is performed using forward and reverse primer pairs specific for hybridizing to a nucleic acid sequence of the cDNA.

15. The method of claim 13, wherein the step of detecting is performed using at least one probe that is specific for hybridizing to a nucleic acid sequence of the cDNA, wherein the probe is labeled with a detectable label.

16. The method of claim 13, wherein the step of amplifying is carried out using he following cycling conditions: 10 min at 95° C., followed by 50 cycles of 15 seconds at 95° C., 1 min at 65° C., followed by 5 s at 95° C. and 1 min at 55° C.
